(19) United States
(12) Patent Application Publication
(10) Pub. No.: US 2022/0152369 A1
(43) Pub. Date: May 19, 2022

(54) WEARABLE SYSTEM AND METHOD FOR GAS DELIVERY TO EXTERIOR SURFACE OF AN EYE

(71) Applicant: Giner Life Sciences, Inc., Newton, MA (US)

(72) Inventors: Melissa N. Schwenk, Somerville, MA (US); Douglas W. Lawrence, Framingham, MA (US); Simon G. Stone, Arlington, MA (US); Griffin James Marella, Somerville, MA (US); Linda A. Tempelman, Lincoln, MA (US)

(21) Appl. No.: 17/530,401

(22) Filed: Nov. 18, 2021

Related U.S. Application Data
(60) Provisional application No. 63/115,374, filed on Nov. 18, 2020.

(51) Int. Cl. A61M 35/00

(52) U.S. Cl. CPC A61M 35/10; A61M 2210/0612; A61M 2202/0208

(57) ABSTRACT

Wearable system and method for gas delivery to the exterior surface of an eye. In one embodiment, the system may include a gas source and a wearable gas delivery device. The gas source may include a housing, one or more electrochemical gas generating devices positioned within the housing, and a control unit for controlling the operation of the one or more electrochemical gas generating devices. The one or more electrochemical gas generating devices may include an electrochemical oxygen concentrator and a water electrolyzer. Oxygen outputted from the electrochemical oxygen concentrator may be combined with oxygen and/or hydrogen outputted from the electrolyzer to produce a therapeutic gas having an enriched oxygen concentration and a hydrogen concentration less than 4%. The wearable gas delivery device, which is fluidly coupled to the gas source, may be worn over the eye and may be used to deliver the therapeutic gas to the eye.

### CROSS-REFERENCE TO RELATED APPLICATIONS

The present application claims the benefit under 35 U.S.C. 119(e) of U.S. Provisional Patent Application No. 63/115,374, inventors Melissa N. Schwenk et al., filed Nov. 18, 2020, the disclosure of which is incorporated herein by reference in its entirety.

### BACKGROUND OF THE INVENTION

The present invention relates generally to methods and systems directed at the healing of eye wounds and relates more particularly to a novel method and system for use in promoting the healing of eye wounds.

Typically, after a patent undergoes corneal surgery, a clear, perforated eye shield (or eye patch) is placed over the patient's eye. The purpose of such a shield is two-fold, namely, to prevent the patient from rubbing the eye, thereby disturbing the wound site, and to allow ambient air to flow to the exterior surface of the eye so that oxygen present in the ambient air may reach the wound site, thereby promoting healing of the wound.

One shortcoming identified by the present inventors with conventional eye shields of the type described above is that the perforations in the eye shield are susceptible to allowing dust and other irritants to pass therethrough and to reach the exterior surface of the eye, thereby causing patient discomfort.

Another shortcoming identified by the present inventors with conventional eye shields of the type described above is that, at best, such shields simply allow ambient air to reach the exterior surface of the eye. By contrast, the present inventors believe that improved wound healing may be effected by administering to the eye a therapeutic gas that differs in composition from ambient air.

Accordingly, there is a need for an eye shield that enables gas, which may be, but is not limited to, a therapeutic gas, to be delivered to the wound site to promote wound healing and that does not require the presence of perforations in the eye shield for the delivery of such gas.

### SUMMARY OF THE INVENTION

It is an object of the present invention to provide a novel system for use in promoting the healing of certain types of eye wounds, such as, but not limited to, corneal surgery wounds and other types of wounds in exterior parts of the eye.

It is another object of the present invention to provide a system as described above that addresses at least some of the shortcomings associated with existing approaches to promoting the healing of such eye wounds.

It is still another object of the present invention to provide a system as described above that is compact, that has a minimal number of parts, that is relatively inexpensive to manufacture, and that is easy to wear and to operate.

It is still yet another object of the present invention to provide a system as described above that functions by delivering a therapeutic gas to the exterior of an eye.

Therefore, according to one aspect of the invention, there is provided a system for delivering a therapeutic gas to the exterior of an eye, the system comprising (a) a gas source for supplying a treatment gas; and (b) a gas delivery device, the gas delivery device being wearable over and substantially covering an exterior of an eye, the gas delivery device being fluidly coupled to the gas source to receive the treatment gas from the gas source, the gas delivery device comprising at least one gas outlet for use in delivering the treatment gas to the exterior of the eye.

In a more detailed feature of the invention, the gas source may comprise an electrochemical gas generating device.

In a more detailed feature of the invention, the electrochemical gas generating device may comprise at least one of a water electrolyzer and an electrochemical oxygen concentrator.

In a more detailed feature of the invention, the electrochemical gas generating device may comprise both a water electrolyzer and an electrochemical oxygen concentrator.

In a more detailed feature of the invention, the treatment gas may comprise at least one of oxygen and hydrogen.

In a more detailed feature of the invention, the treatment gas may comprise oxygen at a concentration above that in ambient air.

In a more detailed feature of the invention, the treatment gas may comprise oxygen at a concentration below pure oxygen.

In a more detailed feature of the invention, the treatment gas may comprise both oxygen and hydrogen.

In a more detailed feature of the invention, the treatment gas may comprise hydrogen at a concentration below 4% by volume.

In a more detailed feature of the invention, the treatment gas may comprise oxygen but not hydrogen.

In a more detailed feature of the invention, the treatment gas may comprise hydrogen but not oxygen.

In a more detailed feature of the invention, the gas delivery device may comprise an eye shield.

In a more detailed feature of the invention, the gas source may comprise a housing wearable over an ear.

In a more detailed feature of the invention, the gas source may comprise a housing adapted to be clipped onto goggles.

In a more detailed feature of the invention, the gas delivery device may comprise goggles.

In a more detailed feature of the invention, the goggles may comprise fluid conduits, and the gas source may be mountable on the goggles in fluid communication with the fluid conduits.

In a more detailed feature of the invention, the gas delivery device may comprise a contact lens bandage.

In a more detailed feature of the invention, the gas delivery device may comprise an eye shield and a foam gasket, and the eye shield may have an inlet for receiving the therapeutic gas from the gas source.

In a more detailed feature of the invention, the gas delivery device may comprise an eye shield and a foam gasket, and the foam gasket may have an inlet for receiving the therapeutic gas from the gas source.

According to another aspect of the invention, there is provided a device for generating a therapeutic gas, the device comprising (a) a housing; (b) an electrochemical oxygen generating device disposed within the housing; and (c) a conditioning unit disposed within the housing for diluting oxygen generated by the electrochemical oxygen generating device.

In a more detailed feature of the invention, the electrochemical oxygen generating device may comprise a water electrolyzer.

In a more detailed feature of the invention, the electrochemical oxygen generating device may comprise an electrochemical oxygen concentrator.

In a more detailed feature of the invention, the conditioning unit may comprise a length of gas permeable tubing.

In a more detailed feature of the invention, the gas permeable tubing may increase in gas permeability when stretched, and said conditioning unit further may further comprise a stretcher for the gas permeable tubing.

According to a further aspect of the invention, there is provided a device for generating a therapeutic gas, the device comprising (a) a housing; (b) an electrochemical oxygen concentrator disposed within the housing; (c) an electrolyzer disposed within the housing; and (d) a mixing chamber for mixing outputs of the electrochemical oxygen concentrator and the electrolyzer.

The present invention is also directed to methods of making and using the above-described system and device.

Additional objects, as well as aspects, features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. The drawings are not necessarily drawing to scale, and certain components may have undersized and/or oversized dimensions for purposes of explication. In the drawings wherein like reference numeral represents like parts:

FIG. 1 is a top view of a first embodiment of a system constructed according to the present invention for delivering a therapeutic gas to the exterior of an eye, the system being designed to be worn over the right eye of a person;

FIG. 2 is a partly exploded perspective view of the system of FIG. 1;

FIG. 3 is an enlarged section view of the electrochemical gas generator shown in FIG. 2;

FIG. 4 is an enlarged perspective view of the gas delivery device of FIG. 1;

FIG. 5 is a bottom view showing the gas delivery device shown in FIG. 1 joined to the tubing shown in FIG. 1;

FIG. 6 is a top view of the gas delivery device and tubing shown in FIG. 5;

FIG. 7 is a section view taken along line 7-7 of FIG. 6;

FIG. 8 is a proximal end view of the shield and the tubing shown in FIG. 6;

FIG. 9 is a section view taken along line 9-9 of FIG. 8;

FIG. 10 is a top view of a second embodiment of a system constructed according to the present invention for delivering a therapeutic gas to the exterior of an eye, the system being designed to be worn over the left eye of a person (the gas source not being shown);

FIG. 11 is a section view taken along line 11-11 of FIG. 10;

FIG. 12 is a bottom view of the system shown in FIG. 10;

FIG. 13 is an exploded perspective view of the system shown in FIG. 10;

FIG. 14 is a fragmentary front view showing the system of FIG. 10 worn by a person;

FIGS. 15 through 17 are top, section and exploded perspective views, respectively, of a third embodiment of a system constructed according to the present invention for delivering a therapeutic gas to the exterior of an eye, the system being designed to be worn over the left eye of a person (the gas source not being shown);

FIG. 18 is a top view, partly in section, of the system shown in FIG. 15, with certain layers of the gas delivery device not being shown;

FIG. 19 is a fragmentary front view of a fourth embodiment of a system constructed according to the present invention for delivering a therapeutic gas to the exterior of an eye, the system being designed to be worn over the left eye of a person and with the gas source held in place with a headband;

FIG. 20 is an enlarged perspective view showing the gas source of FIG. 19;

FIG. 21 is a fragmentary front view of a fifth embodiment of a system constructed according to the present invention for delivering a therapeutic gas to the exterior of an eye, the system being designed to be worn over the left eye of a person and with the gas source held in place with glasses;

FIGS. 22 and 23 are enlarged perspective and end views, respectively, showing the gas source of FIG. 21;

FIGS. 24A through 24H are various views of a sixth embodiment of a system constructed according to the present invention for delivering a therapeutic gas to the exterior of an eye, the system being designed to be worn like a pair of goggles;

FIG. 25 is a perspective view of a seventh embodiment of a system constructed according to the present invention for delivering a therapeutic gas to the exterior of an eye, the system being designed to be worn directly over the eye of a person;

FIGS. 26 through 33 schematic representations of alternative embodiments of a device for electrochemically generating a therapeutic gas;

FIGS. 34 through 36 are schematic representations of alternative embodiments of a conditioning unit for diluting the concentration of a gas;

FIGS. 37A and 37B are alternative embodiments of a gas permeable tubing assembly suitable for use in the conditioning device of FIG. 36;

FIGS. 38 through 40 are additional alternative embodiments of a conditioning unit for diluting the concentration of a gas;

FIGS. 41A and 41B are perspective and top views, respectively, of an alternative embodiment of a housing for a device for electrochemically generating a therapeutic gas;

FIG. 42 is a perspective view of another alternative embodiment of a housing for a device for electrochemically generating a therapeutic gas, the housing being shown with a length of tubing;

FIG. 43 is a graph depicting the oxygen partial pressure measured and predicted in the Example by dilution of varying inlet flows to a silicone tube with ambient air on the outside (with solid dots representing measured values and the line showing the predicted trend); and

FIG. 44 is a graph depicting the partial pressure of oxygen when a permeation tube is at 100% length (closed circles) vs 150% length (open circles) for a variety of flow rates as discussed in the Example.

### DETAILED DESCRIPTION OF THE INVENTION

As noted above, the present invention is directed at a novel method and system for use in promoting the healing of certain types of eye wounds, such as, but not limited to, those resulting from corneal surgery. More specifically, said method and system may involve delivering a therapeutic gas to the exterior of an eye using a wearable delivery device, such as, but not limited to, an eye patch, a pair of goggles, or the like. The method and system of generating a therapeutic gas could be applied to the treatment of an anatomical site or organ that has a wound or other dysfunction other than the eye.

In contrast with existing approaches to promoting the healing of corneal surgery wounds and similar eye wounds, wherein such approaches include the wearing of a perforated eye patch that allows ambient air to pass through the eye patch, the present invention does not require the use of a perforated eye patch nor does the present invention require the delivery of ambient air. Instead, as will be described further below, the present invention preferably utilizes a gas source to supply one or more therapeutic gases (e.g., oxygen gas; hydrogen gas; a mixture of oxygen gas and hydrogen gas; one or more of humidified oxygen gas and humidified hydrogen gas), and some or all of these gases may be delivered to an eye using a novel wearable delivery device that is positioned over the exterior of the eye.

More specifically, in one embodiment, the gas source may comprise an electrochemical device or other device for generating, in situ, the one or more therapeutic gases. In situ generation includes, but is not limited to, devices that use electrochemical, chemical, physical (e.g., molecular sieve) techniques or a combination of these techniques. In another embodiment, the gas source may comprise a container holding a preloaded quantity of one or more gases.

Electrochemical devices are particularly well-suited for the generation and delivery of one or more product gases at a controlled dose per unit time. In the present invention, which preferably involves the delivery of one or more electrochemically generated gases, and, in at least some embodiments, involves the delivery of electrochemically generated oxygen, such oxygen may be electrochemically generated via one of the following two types of reactions: (i) water electrolysis; and (ii) electrochemical oxygen concentration. In those embodiments that additionally or alternatively involve the delivery electrochemically generated hydrogen, such hydrogen may also be electrochemically generated via water electrolysis.

Water electrolysis is a common technique for generating oxygen and hydrogen and typically involves using an electrical current to convert water into gaseous oxygen and gaseous hydrogen. One way to perform water electrolysis is with a proton exchange membrane (PEM) electrolyzer. A PEM electrolyzer typically comprises a proton exchange membrane (PEM), an anode with catalyst on one face of the PEM, and a cathode with catalyst on the opposite face of the PEM, the combination of the PEM, the anode and the cathode often referred to as a membrane electrode assembly (MEA). The PEM, itself, typically comprises an ion-exchange polymer which, when humidified, allows the migration of protons therethrough. The PEM ion-exchange polymer also substantially prevents reactants and products at each electrode from mixing. In use, power is consumed to split water molecules on one side of the MEA to form oxygen gas and protons. The protons migrate through the MEA to the other side, where they combine with electrons to form hydrogen gas. The oxygen production rate for a PEM electrolyzer is governed by and proportional to the electrical current provided and can be tailored for many applications. Water electrolysis may be desirable in certain cases as a production technique due to its high process efficiency, its product selectivity, and its inherent ability to control production rate by controlling the applied current.

Electrochemical oxygen concentration involves using an electrical current to concentrate oxygen present in air to pure oxygen. An electrochemical device designed for electrochemical oxygen concentration is often referred to as an electrochemical oxygen concentrator and may also comprise an MEA. In operation, an MEA-based electrochemical oxygen concentrator consumes electrical current to convert ambient oxygen to water at the cathode side of an MEA. The water product of this cathodic reaction then diffuses through the MEA to the anode, where water is oxidized into oxygen. The pure oxygen generated at the anode is then directed out of the electrochemical oxygen concentrator, where it can be used. The protons from the oxidized water at the anode cross the MEA again to the cathode to combine with oxygen from the air to form water vapor, whereupon the process repeats itself. The proton exchange membrane of the MEA also comprises an ion-exchange polymer which, when humidified, allows the migration of protons. The ion-exchange polymer also prevents reactants and products at each electrode from mixing, and other gases found in the ambient environment, such as nitrogen, from contaminating the pure oxygen product. The oxygen concentration rate is governed by and proportional to the electrical current provided and can be tailored for many applications.

In many instances, an electrochemical device capable of generating oxygen may alternatively use either water electrolysis or electrochemical oxygen concentration at a given time, depending on the reactants available and/or voltage and current settings, and such an electrochemical device may be tailored to be more appropriate for one reaction over the other.

As will be discussed further below, one aspect of the present invention is that a therapeutic gas comprising one or more constituent gases may be delivered to an eye. For example, where oxygen is delivered to the eye, such oxygen may promote wound healing. As another example, where hydrogen is delivered to the eye, such hydrogen may have anti-inflammatory, antioxidant and/or antiapoptotic effects. Where, for example, oxygen and/or hydrogen are delivered to the eye, such oxygen and/or hydrogen may be generated electrochemically, for example, by the hydrolysis of water. Such water may include water that is present in the ambient environment. Alternatively, where, for example, oxygen is delivered to the eye, such oxygen may be generated electrochemically by the concentration of oxygen from air or oxygen-enriched air. Such air or oxygen-enriched air may include air that is present in the ambient environment and/or oxygen-enriched air that was previously generated.

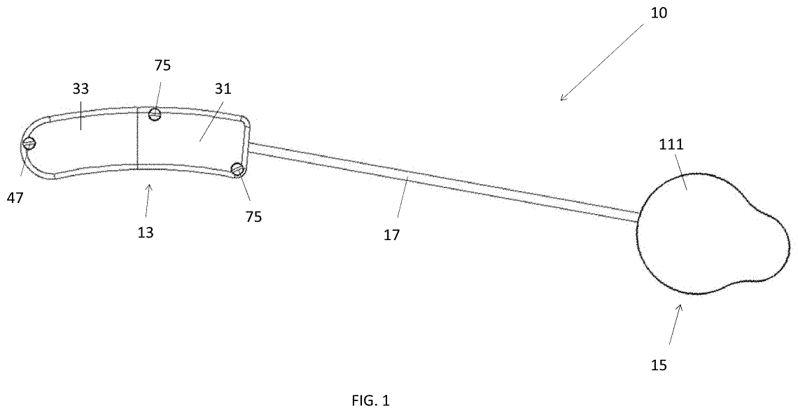

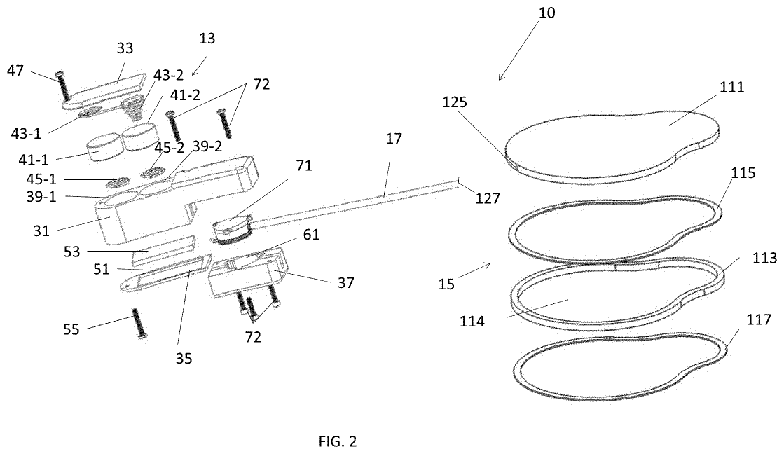

Referring now to FIGS. 1 and 2, there are shown various views of a first embodiment of a system for delivering a therapeutic gas to the exterior of an eye, the system being constructed according to the present invention and represented generally by reference numeral 10. (For simplicity and clarity, certain components of system 10 that are not critical to the understanding of the present invention are either not shown or described herein or are shown and/or described herein in a simplified manner.) For illustrative purposes, system 10 is shown in FIGS. 1 and 2 designed for use over a right eye of a person. However, it can readily be appreciated that system 10 could easily be modified to be used over a left eye.

System 10 may comprise a gas source 13, a gas delivery device 15, and a length of tubing 17.

Gas source 13, in turn, may comprise a housing 25. Housing 25, which may be appropriately dimensioned to be worn on the exterior of a right ear (e.g., over or behind the right ear) may be collectively formed by a battery storage member 31, a top cover 33, a bottom cover 35, and an electrochemical gas generator storage member 37. One or more, and preferably all, of battery storage member 31, top cover 33, bottom cover 35, and electrochemical gas generator storage member 37 may be made of or comprise one or more suitably strong, rigid, and biocompatible materials, such as, but not limited to, acrylic, titanium, acetal resin (e.g., DELRIN® acetal homopolymer (polyoxymethylene (POM)), DuPont de Nemours, Inc., Wilmington, Del.), and the like, and may be formed by machining, molding, 3D printing, and/or any other suitable manufacturing technique.

Battery storage member 31 may be shaped to include cavities 39-1 and 39-2. Cavity 39-1 may be dimensioned to removably receive a battery 41-1, a top contact 43-1, and a bottom contact 45-1, and cavity 39-2 may be dimensioned to removably receive a battery 41-2, a top contact 43-2, and a bottom contact 45-2. Batteries 41-1 and 41-2, which may be used to power an electrochemical gas generator to be described below, may be a primary or rechargeable battery or may be any other type of similarly suitable power source. Although batteries 41-1 and 41-2 are shown as having a generally cylindrical shape, it is to be understood that the shape of batteries 41-1 and 41-2 can be cuboid or any other suitable shape. Acceptable battery chemistries and battery packagings may be any that are safe for use near the ear and face. Acceptable batteries may include, but are not limited to, zinc-air primary batteries of the type that are commonly used in hearing aids. Batteries 41-1 and 41-2 may be replaced or recharged during patient use. Batteries 41-1 and 41-2 may include energy harvesting (also called ambient energy) technologies from the wearable electronics field.

Top cover 33, which may be used to cover the tops of cavities 39-1 and 39-2, may be secured to a top end of battery storage member 31 using a screw 47. Bottom cover 35, which may include a recess 51 for receiving a printed circuit board 53 with control electronics, may be secured to the bottom of battery storage member 31 using a screw 55.

Electrochemical gas generator storage member 37 may be shaped to include a cavity 61. Cavity 61, in turn, may be used to receive an electrochemical gas generator 71, which will be further described below. Electrochemical gas generator storage member 37 may be secured to battery storage member 31 using screws 72. Also, although not shown, one or more ambient reactant delivery tubes or lumens may be appropriately provided to permit ambient air to gain access to the operative components of electrochemical gas generator 71.

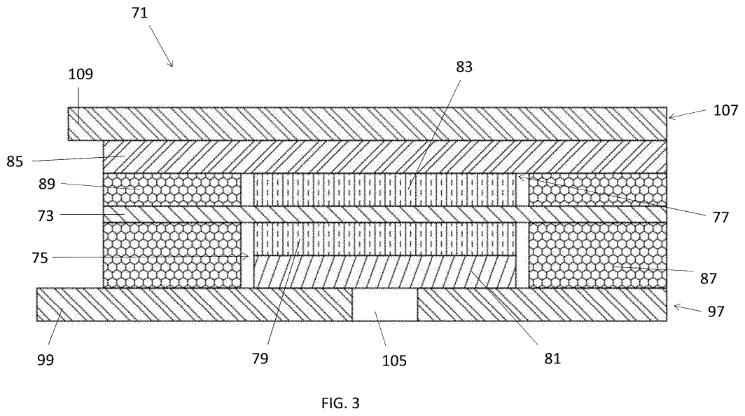

Referring now to FIG. 3, electrochemical gas generator 71 is shown in greater detail. Electrochemical gas generator 71 may be operated as a water electrolyzer to generate both oxygen gas and hydrogen gas or, alternatively, may be operated as an electrochemical oxygen concentrator to generate oxygen gas. Electrochemical gas generator 71 may comprise a solid polymer electrolyte membrane (PEM) 73 (also known in the art as a proton exchange membrane). PEM 73 is preferably a non-porous, ionically-conductive, electrically-non-conductive, liquid permeable and substantially gas-impermeable membrane. PEM 73 may consist of or comprise a homogeneous perfluorosulfonic acid (PFSA) polymer. Said PFSA polymer may be formed by the copolymerization of tetrafluoroethylene and perfluorovinylether sulfonic acid. See e.g., U.S. Pat. No. 3,282,875, inventors Connolly et al., issued Nov. 1, 1966; U.S. Pat. No. 4,470,889, inventors Ezzell et. al., issued Sep. 11, 1984; U.S. Pat. No. 4,478,695, inventors Ezzell et. al., issued Oct. 23, 1984; and U.S. Pat. No. 6,492,431, inventor Cisar, issued Dec. 10, 2002, all of which are incorporated herein by reference in their entireties. A commercial embodiment of a PFSA polymer electrolyte membrane is manufactured by The Chemours Company FC, LLC (Fayetteville, N.C.) as NAFION™ extrusion cast PFSA polymer membrane.

PEM 73 may be a generally planar unitary structure in the form of a continuous film or sheet. In the present embodiment, when viewed from above or below, PEM 73 may have a general circular shape. Moreover, the overall shape of electrochemical gas generator 71, when viewed from above or below, may correspond generally to the shape of PEM 73. However, it is to be understood that PEM 73, as well as electrochemical gas generator 71 as a whole, is not limited to a generally circular shape and may have a generally rectangular, annular, or other suitable shape.

Electrochemical gas generator 71 may further comprise an anode 75 and a cathode 77. Anode 75 and cathode 77 may be positioned along two opposing major faces of polymer electrolyte membrane 73. In the present embodiment, anode 75 is shown positioned along the bottom face of PEM 73, and cathode 77 is shown positioned along the top face of PEM 73; however, it is to be understood that the positions of anode 75 and cathode 77 relative to PEM 73 could be reversed.

Anode 75, in turn, may comprise an anode electrocatalyst layer 79 and an anode support 81. Anode electrocatalyst layer 79 may be positioned in direct contact with PEM 73, and, in the present embodiment, is shown as being positioned directly below and in contact with the bottom side of PEM 73. Anode electrocatalyst layer 79 defines the electrochemically active area of anode 75 and preferably is sufficiently porous and electrically- and ionically-conductive to sustain a high rate of surface oxidation reaction. Anode electrocatalyst layer 79, which may be an anode electrocatalyst layer of the type conventionally used in a PEM-based water electrolyzer, may comprise electrocatalyst particles in the form of a finely divided electrically-conductive and, optionally, ionically-conductive material (e.g., a metal powder) which can sustain a high rate of electrochemical reaction. The electrocatalyst particles may be distributed within anode electrocatalyst layer 79 along with a binder, which is preferably ionically-conductive, to provide mechanical fixation.

Anode support 81, which may be an anode support of the type conventionally used in a PEM-based water electrolyzer and may be, for example, a film or sheet of porous titanium, preferably is sufficiently porous to allow fluid (gas and/or liquid) transfer between anode electrocatalyst layer 79 and a fluid cavity external to electrochemical gas generator 71. To this end, anode support 81 may have pore sizes on the order of, for example, approximately 0.001-0.5 mm. Anode support 81 may also contain macroscopic channel features, for example, on the order of 0.2-10 mm to further assist in fluid distribution. In addition, anode support 81 is preferably electrically-conductive to provide electrical connectivity between anode electrocatalyst layer 79 and an anode current collector to be discussed below. Anode support 81 is also preferably ionically-non-conductive. Anode support 81 may be positioned in direct contact with anode electrocatalyst layer 79 and, in the present embodiment, is shown as being positioned directly below anode electrocatalyst layer 79 such that anode electrocatalyst layer 79 may be sandwiched between and in contact with PEM 73 and anode support 81. Anode support 81 may be dimensioned to entirely cover a surface (e.g., the bottom surface) of anode electrocatalyst layer 79, and, in fact, anode 75 may be fabricated by depositing anode electrocatalyst layer 79 on anode support 81.

Cathode 77 may comprise a cathode electrocatalyst layer 83 and a cathode support 85. Cathode electrocatalyst layer 83 may be positioned in direct contact with PEM 73, and, in the present embodiment, is shown as being positioned directly above and in contact with the top of PEM 73. Cathode electrocatalyst layer 83 defines the electrochemically active area of cathode 77 and preferably is sufficiently porous and electrically- and ionically-conductive to sustain a high rate of surface reduction reaction. Cathode electrocatalyst layer 83, which may be a cathode electrocatalyst layer of the type conventionally used in a PEM-based water electrolyzer, may comprise electrocatalyst particles in the form of a finely divided electrically-conductive and, optionally, ionically-conductive material (e.g., a metal powder) which can sustain a high rate of electrochemical reaction. The electrocatalyst particles may be distributed within cathode electrocatalyst layer 83 along with a binder, which is preferably ionically-conductive, to provide mechanical fixation. The reactants and products involved at anode 75 and cathode 77 may implicate ionic species that are mobile throughout the electroactive surface; therefore, an ionically-conductive medium comprising PEM 73 and, optionally, one or more ionically-conductive catalyst binders in electrocatalyst layers 79 and 83 may couple the electrodes and may allow ions to flow in support of the overall reaction electrochemistry.

Cathode support 85, which may be a cathode support of the type conventionally used in a PEM-based water electrolyzer and may be, for example, a film or sheet of porous carbon, preferably is sufficiently porous to allow fluid (gas and/or liquid) transfer between cathode electrocatalyst layer 83 and a fluid cavity external to electrochemical gas generator 71. To this end, cathode support 85 may have pore sizes on the order of, or example, approximately 0.001-0.5 mm Cathode support 85 may also contain macroscopic channel features, for example, on the order of 0.2-10 mm to further assist in fluid distribution. In addition, cathode support 85 is electrically-conductive to provide electrical connectivity between cathode electrocatalyst layer 83 and a cathode current collector to be discussed below. Cathode support 85 is also preferably ionically-non-conductive. Cathode support 85 may be positioned in direct contact with cathode electrocatalyst layer 83 and, in the present embodiment, is shown as being positioned directly above cathode electrocatalyst layer 83 such that cathode electrocatalyst layer 83 may be sandwiched between and in contact with PEM 73 and cathode support 85. Cathode support 85 may be dimensioned to entirely cover a surface (e.g., the top surface) cathode electrocatalyst layer 83, and, in fact, cathode 77 may be fabricated by depositing cathode electrocatalyst layer 83 on cathode support 85.

The combination of PEM 73, anode 75, and cathode 77, or the combination of PEM 73, anode electrocatalyst layer 79, and cathode electrocatalyst layer 83 may be regarded collectively as a membrane-electrode assembly (MEA).

Electrochemical gas generator 71 may further comprise an anode seal 87 and a cathode seal 89. Anode seal 87, which may be an anode seal of the type conventionally used in a PEM-based water electrolyzer, may be a generally annular or frame-like member mounted around the periphery of anode 75 in a fluid-tight manner. (Anode seal 87 may be positioned in direct contact with the periphery of anode 75 or there may be a small gap between anode seal 87 and the periphery of anode 75 to facilitate assembly.) Anode seal 87, which may be made of polytetrafluoroethylene (PTFE), ethylene-propylene-diene-monomer (EPDM) rubber, or another similarly suitable material, may be ionically-non-conductive and electrically non-conductive. Anode seal 87 may also be non-porous and fluid-impermeable.

Cathode seal 89, which may be a cathode seal of the type conventionally used in a PEM-based water electrolyzer, may be a generally annular or frame-like member mounted around the periphery of cathode 77 in a fluid-tight manner. (Cathode seal 89 may be positioned in direct contact with the periphery of cathode 77 or there may be a small gap between cathode seal 89 and the periphery of cathode 77 to facilitate assembly.) Cathode seal 89, which may be made of polytetrafluoroethylene (PTFE), ethylene-propylene-diene-monomer (EPDM) rubber, or another similarly suitable material, may be ionically-non-conductive and electrically-non-conductive. Cathode seal 89 may also be non-porous and fluid-impermeable.

In the present embodiment, anode 75 and anode seal 87 may be dimensioned to jointly match the footprint of the bottom surface of PEM 73. In addition, cathode support 85, cathode catalyst layer 83, and cathode seal 89 may also be dimensioned to jointly match the footprint of the top surface of PEM 73. Notwithstanding the above, it is to be understood that the footprints of the foregoing components may be varied from what is described above.

Electrochemical gas generator 71 may further comprise an anode current collector 97. Anode current collector 97 may be similar to an anode current collector of the type conventionally used in a PEM-based water electrolyzer and may comprise, for example, a platinum-coated titanium sheet. When viewed from below, anode current collector 97 may have a footprint that substantially matches the collective footprints of anode 75 and anode seal 87, except that anode current collector 97 may additionally comprise a tab 99 that may extend radially outwardly a short distance beyond the periphery of anode seal 87 and that may be used as a terminal. Anode current collector 97 may also comprise a through hole 105, which may be used to receive the proximal end of tubing 17.

Electrochemical gas generator 71 may further comprise a cathode current collector 107, which may comprise a cathode current collector of the type conventionally used in a PEM-based water electrolyzer and may be, for example, a platinum-coated titanium sheet. When viewed from below, cathode current collector 107 may have a footprint that substantially matches the collective footprints of cathode 77 and cathode seal 89, except that cathode current collector 107 may additionally comprise a tab 109 that may extend radially outwardly a short distance beyond the footprint of cathode seal 89 and that may be used as a terminal.

Although not shown, electrochemical gas generator 71 may further comprise other components commonly found in conventional PEM-based water electrolyzers. For example, the static forces upon electrochemical gas generator 71 that may be required to compress anode seal 87 and cathode seal 89 to sustain good electrical contact of the serial components of electrochemical gas generator 71 and to achieve good sealing of the cell perimeter may be established and maintained using a variety of conventional fixturing or joining implements and techniques about the internal or external periphery of the assembly. Such implements may include, for instance, fasteners (e.g., screws, rivets, etc.) which may clamp endplates at either end of the serial components, or adhesives, cements, or welds which cohere the elements together in the seal region. Such implements and techniques are known to those of ordinary skill in the art. Electrochemical gas generator 71 may be operated at a range of currents, voltages and flow rates as is possible with an electrochemical oxygen generator and may be operated continuously or intermittently or via a feedback control mechanism to meet the needs of the application.

Electrochemical gas generator 71 may be designed to be particularly amenable to oxygen delivery to the eye. For such a design, it may be important to have a suitable oxygen delivery rate to mitigate the possible effect of over-drying the surface of the eye, as well as to reduce irritation caused by excess gas flow. Gas source 13 may include structure to ensure the proper routing of gas streams from the ambient air to and from anode 75 and cathode 77 and to and from the eye. Proper routing may provide optimal use of the gas streams as reactants and as a treatment for eye conditions. Optimal use may include provision of optimal pO2, humidity, sterility, and energy use. The control electronics for electrochemical gas generator 71 may precisely set the current of electrochemical gas generator 71 based on the application's specific flow rate.

The use of electrochemical gas generator 71 is preferred over the use of ambient oxygen for at least the reasons that electrochemical gas generator 71 provides a settable flow rate, a pure oxygen stream, a lack of particulate or contaminants in the product gas stream, and an ability to reach higher than ambient concentrations of oxygen. As can be appreciated, based on the application, the length of time electrochemical gas generator 71 would be operated may vary, as may its flow rate.

Referring back now to FIGS. 1 and 2, tubing 17 may be an elongated flexible, yet supportive, structure made of or comprising one or more suitable chemically inert, biocompatible materials. A proximal end of tubing 17 may be permanently or removably coupled to an output of electrochemical gas generator 71, and a distal end of tubing 17 may be permanently or removably coupled to gas delivery device 15. In this manner, as will be discussed further below, a product gas from electrochemical gas generator 71 may be delivered through tubing 17 and gas delivery device 15 to the wearer. Tubing 17 may be equipped with filters and/or check valves (not shown) to prevent electrochemical gas generator 71 from becoming contaminated by biological materials or from condensate flow backwards into electrochemical gas generator 71.

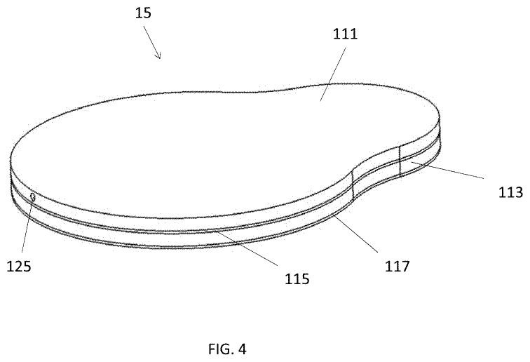

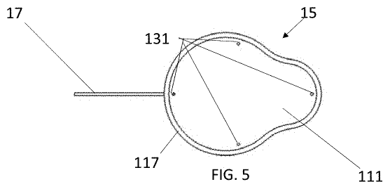

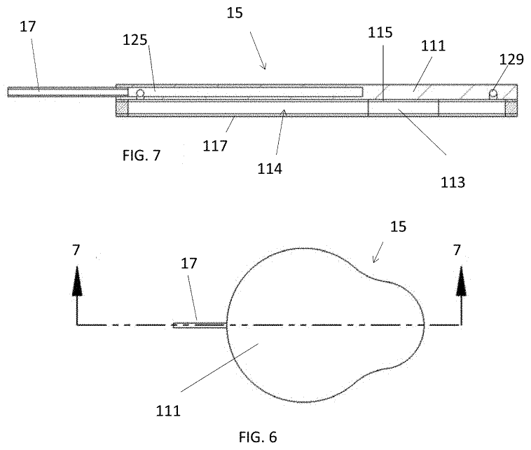

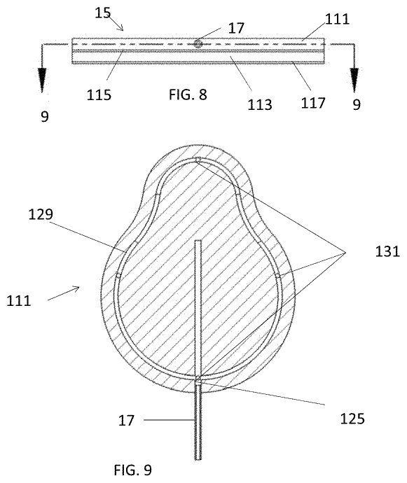

Referring now to FIGS. 1-2 and 4-9, gas delivery device 15 may comprise an eye shield 111. Eye shield 111, which may be a unitary member or a multi-piece structure, may have a pear-shaped or other suitably shaped footprint appropriately dimensioned to completely cover an eye. Eye shield 111 may be custom fitted to the wearer's orbital bones or may have a standard shape. Eye shield 111 may be made of cloth, plastic (e.g., acrylic, polycarbonate, vinyl), or any other one or more suitable, biocompatible materials. Eye shield 111 may be machined, molded, 3D printed, or otherwise manufactured in any suitable way depending on the material. Eye shield 111 may be optically clear, transparent, and translucent, or may be tinted to varying degrees of transparency and translucence.

Gas delivery device 15 may further comprise a foam gasket 113. Foam gasket 113, which may be a unitary member made of a medical grade foam or similar material, may be sized and shaped to have an outer dimension that substantially matches that of eye shield 111 while having an open central portion 114. The top surface of foam gasket 113 may be permanently secured to the bottom surface of eye shield 111 using a first adhesive member 115 having a substantially matching size and shape. A second adhesive member 117 may be secured to the bottom surface of foam gasket 113 and may comprise a suitable non-permanent or repositionable adhesive on its bottom surface so as to permit temporary securement to the skin surrounding the eye. Foam gasket 113 and/or eye shield 111 may be nonporous but yet may have some gas permeability to prevent gas pressure between gas delivery device 15 and the eye from becoming too great.

An axial bore 125 may be provided in eye shield 111 and may extend distally a short distance from a proximal end of eye shield 111. Bore 125 may be appropriately dimensioned to securely receive a distal end 127 of tubing 17. Eye shield 111 may be further shaped to include a continuous inner channel 129. Channel 129 may be spaced sufficiently inwardly from its periphery to be located to the interior relative to foam gasket 113. Channel 129 may be in fluid communication with axial bore 127. Eye shield 111 may further include a plurality of outlets 131 extending straight down from channel 129 to the bottom surface of eye shield 111. In this manner, gas conveyed to eye shield 111 from tubing 17 may be readily distributed throughout the volume interior to foam gasket 113.

It is to be understood that, although bore 125, channel 129, and outlets 131 are formed by removing material from eye shield 111, one or more of these structures could be formed by or additionally include lengths of tubing or the like.

In use, gas source 13 may be mounted over the right ear, gas delivery device 15 may be mounted over the right eye, and gas source 13 may be turned on for desired continuous or intermittent use. The entirety of system 10 may be disposed after a certain period of time or after use by one patient. Alternatively, portions of system 10 may be replaced after time as different portions may be last for different periods of time. If desired, system 10 may be designed to be reusable and may be sterilizable or re-sterilizable.

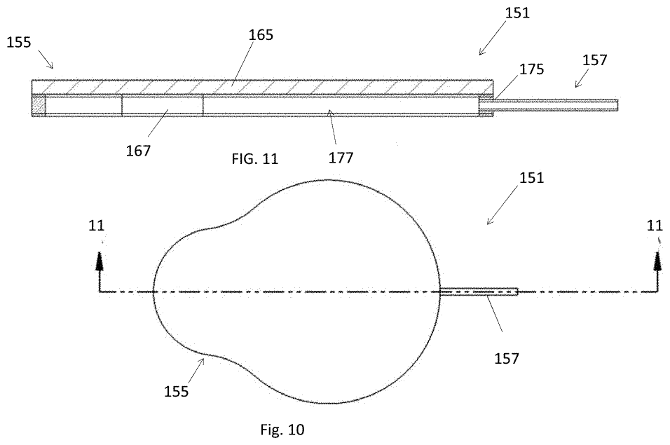

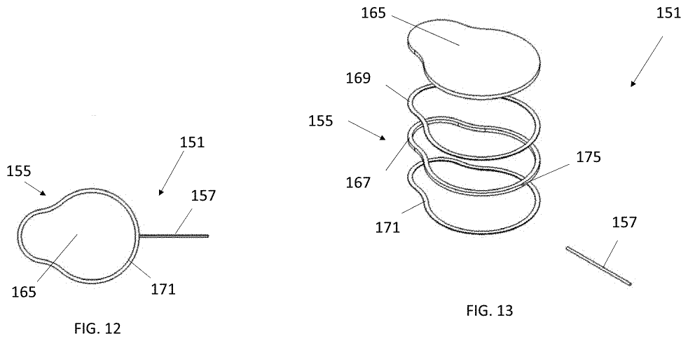

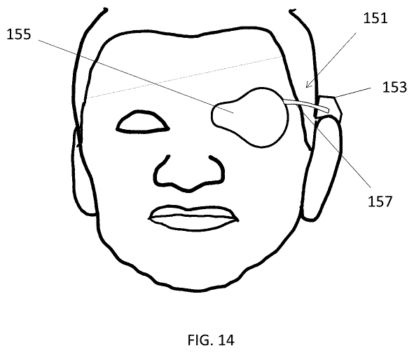

Referring now to FIGS. 10 through 14, there are shown various views of a second embodiment of a system constructed according to the present invention for delivering a therapeutic gas to the exterior of an eye, the system being represented generally by reference number 151. (For simplicity and clarity, certain components of system 151 that are not critical to the understanding of the present invention are either not shown or described herein or are shown and/or described herein in a simplified manner.)

System 151, which may be designed to be worn over the left eye of a person, may comprise a gas source 153, a gas delivery device 155, and a length of tubing 157. Gas source 153 may be similar to gas source 13, except that gas source 153 may be designed to be worn on the left ear of the user, as opposed to the right ear of the user. Tubing 157 may be identical to tubing 17.

Gas delivery device 155 may be similar to gas delivery device 15 and may comprise an eye shield 165, a foam gasket 167, a first adhesive member 169, and a second adhesive member 171. Some of the differences between gas delivery devices 15 and 153 may be that (i) foam gasket 167 may include a bore 175 that corresponds to bore 125 of eye shield 111, wherein bore 175 may be used to receive tubing 157, and (ii) eye shield 165 need not include any structures corresponding to bore 125, channel 129, or outlets 131. As such, when gas is delivered to foam gasket 167, such gas fills the cavity 177 of foam gasket from a single entry point, as opposed to being distributed through a plurality of points, as in system 10. Notwithstanding the above, the numbers of entry points to the cavity of foam gasket 167 of system 151 and to the cavity of foam gasket 113 of system 10 are non-limiting and may be modified as desired.

System 151 may be used analogously to system 10.

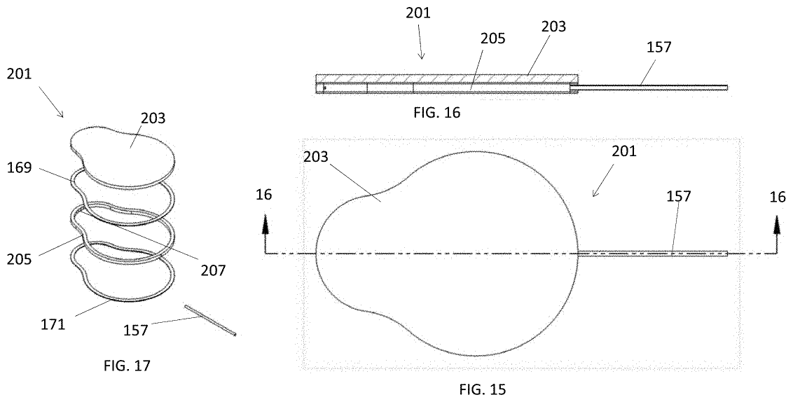

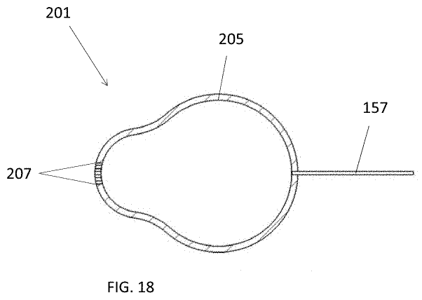

Referring now to FIGS. 15 through 18, there are shown various views of a third embodiment of a system constructed according to the present invention for delivering a therapeutic gas to the exterior of an eye, the system being represented generally by reference number 201. (For simplicity and clarity, certain components of system 201 that are not critical to the understanding of the present invention are either not shown or described herein or are shown and/or described herein in a simplified manner.)

System 201, which may be designed to be worn over the left eye of a person, may be similar to system 151, the principal difference between the two systems being that, whereas eye shield 165 and/or foam gasket 167 of system 151 may be made of one or more materials that are sufficiently gas permeable to prevent the gas pressure over the eye from becoming too great, system 201 may comprise an eye shield 203 and a foam gasket 205 that are made of one or more materials that are substantially gas impermeable or whose gas permeability is insufficiently low to address the aforementioned problem of high gas pressure building up over the eye. Accordingly, as a way of obviating this problem, foam gasket 205 may include one or more small openings 207. It is to be understood that the number and placement of openings 207 in the present embodiment is merely illustrative and that the number and placement of openings 207 may be varied as desired.

It is also to be understood that the precepts of this embodiment may also be applied to system 10.

System 201 may be used analogously to system 10.

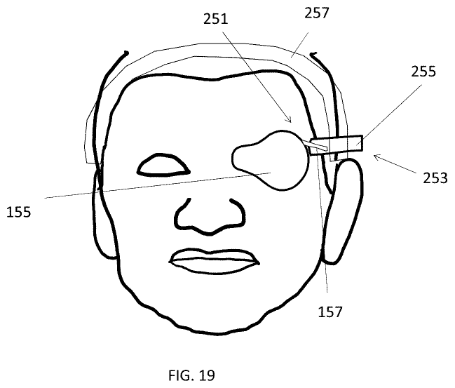

Referring now to FIG. 19, there is shown a fragmentary front view of a fourth embodiment of a system constructed according to the present invention for delivering a therapeutic gas to the exterior of an eye, the system being represented generally by reference number 251. (For simplicity and clarity, certain components of system 251 that are not critical to the understanding of the present invention are either not shown or described herein or are shown and/or described herein in a simplified manner.)

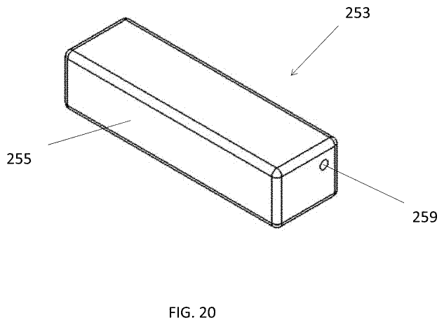

System 251 may be similar in most respects to system 151, the primary difference between the two systems being that whereas system 151 may include a gas source 153 comprising a housing that is designed to be worn on the ear of a user, system 251 may include a gas source 253 comprising a housing 255 that is designed to be kept in place on the head of a user using a headband 257 or similar accessory. Housing 255 of gas source 253 is shown in greater detail in FIG. 20. An opening 259 is provided in housing 255 to receive the proximal end of tubing 157.

It is to be understood that the precepts of system 251 may be applied to the other embodiments disclosed herein.

System 251 may be used analogously to system 10.

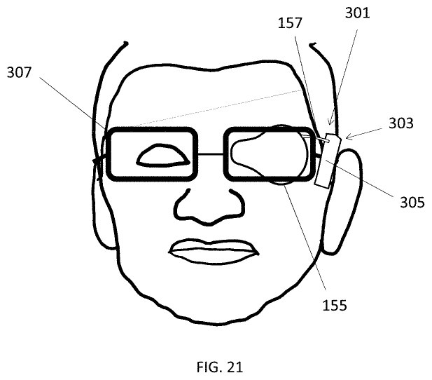

Referring now to FIG. 21, there is shown a fragmentary front view of a fifth embodiment of a system constructed according to the present invention for delivering a therapeutic gas to the exterior of an eye, the system being represented generally by reference number 301. (For simplicity and clarity, certain components of system 301 that are not critical to the understanding of the present invention are either not shown or described herein or are shown and/or described herein in a simplified manner.)

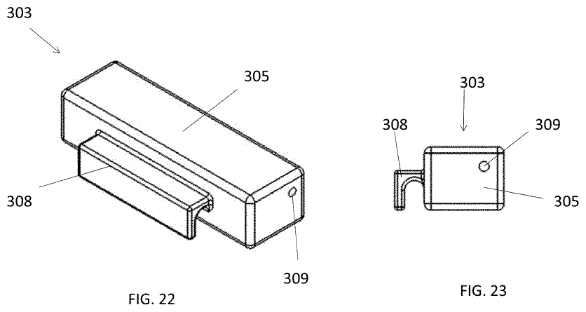

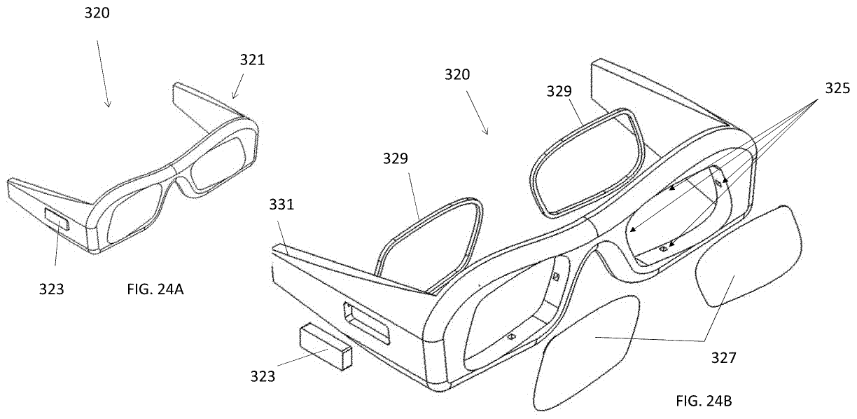

System 301 may be similar in most respects to system 151, the primary difference between the two systems being that whereas system 151 may include a gas source 153 comprising a housing that is designed to be worn on the ear of a user, system 301 may include a gas source 303 comprising a housing 305 that is designed to be kept in place on the head of a user by attachment to glasses 307 via a clip 308. Housing 305 of gas source 303 is shown in greater detail in FIGS. 22 and 23. An opening 309 is provided in housing 305 to receive the proximal end of tubing 157.

It is to be understood that the precepts of system 301 may be applied to the other embodiments disclosed herein.

System 301 may be used analogously to system 10.

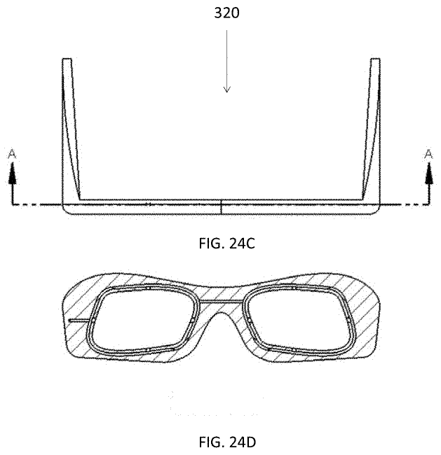

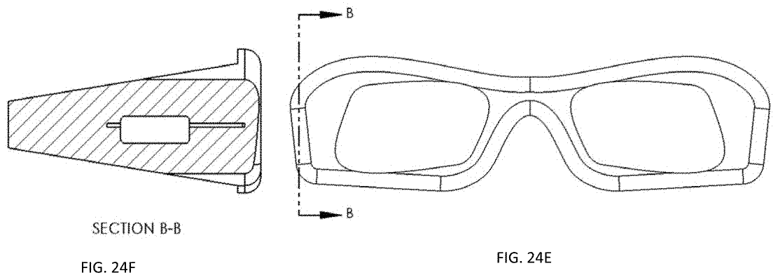

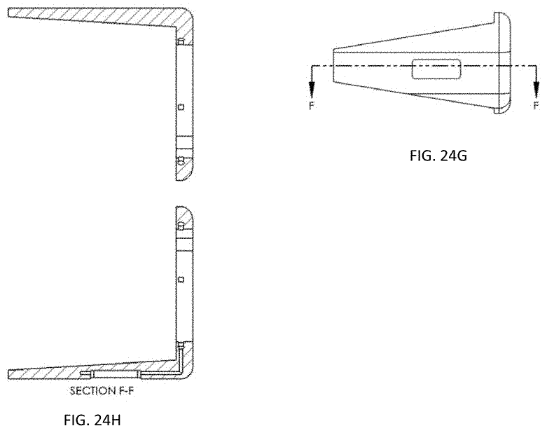

Referring now to FIGS. 24A through 24H, there are shown various views of a sixth embodiment of a system constructed according to the present invention for delivering a therapeutic gas to the exterior of an eye, the system being represented generally by reference number 320. (For simplicity and clarity, certain components of system 320 that are not critical to the understanding of the present invention are either not shown or described herein or are shown and/or described herein in a simplified manner.)

System 320 may comprise a pair of goggles 321 and a gas source 323, wherein gas source 323 may be mounted on goggles 321 and wherein goggles may have integrated fluid conduits to lead gas from gas source 323 to a plurality of outlets 325. Lenses 327 may keep the gas from escaping away from the patient, and sealing foam 329 may be positioned on the interior face of goggles 321.

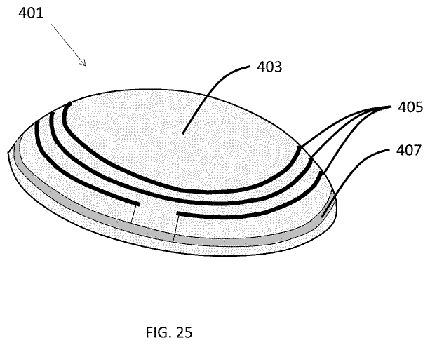

Referring now to FIG. 25, there is shown a sixth embodiment of a system constructed according to the present invention for delivering a therapeutic gas to the exterior of an eye, the system being represented generally by reference number 401. (For simplicity and clarity, certain components of system 401 that are not critical to the understanding of the present invention are either not shown or described herein or are shown and/or described herein in a simplified manner.)

System 401 may be designed to be worn between the eye and one or both of the eyelids, similar to the wearing of a contact lens. Consequently, the eye cover of system 401 may comprise a contact lens bandage 403. An electrochemical gas generator 407 may have a generally annular shape and may be embedded within contact lens bandage 403 for direct delivery of oxygen and/or hydrogen to the eye. Contact lens bandage 403 also may comprise induction coils 405 for transfer of energy from a wearable electronics housing (not shown).

As noted above, in accordance with the present invention, it is possible to electrochemically generate substantially pure oxygen using an electrochemical oxygen concentrator, and it is also possible to electrochemically generate both oxygen and hydrogen using a water electrolyzer. In fact, in some cases, it is possible to selectively operate an electrochemical cell as either an electrochemical oxygen concentrator or as a water electrolyzer. Moreover, where an electrochemical cell is operated as a water electrolyzer, one may use the generated oxygen for therapeutic applications and simply vent or otherwise dispose of the generated hydrogen or vice versa or use both the generated oxygen and the generated hydrogen. Furthermore, it is possible to use two or more electrochemical gas generators, such as a plurality of electrochemical oxygen concentrators, a plurality of water electrolyzers, or some combination of at least one electrochemical oxygen concentrator and at least one water electrolyzer.

In certain applications, particular oxygen and/or hydrogen concentrations may be desired for optimal therapeutic effect. For example, pure (100%) oxygen may present certain hazards to tissues associated with hyperoxic toxicity or flammability whereas 21% oxygen (ambient air) may be insufficient for delivery of sufficient oxygen to respiring tissue. Oxygen concentrations that are intermediate to the aforementioned extremes can be achieved by incorporating a subsystem or component which dilutes the enriched oxygen produced by the electrochemical cell to a concentration greater than 21% and less than 100%. This may be done, for example, by mixing ambient air into the generated oxygen stream between the electrochemical cell and the point of application to the tissue. A device for providing such mixing may be referred to herein as an oxygen conditioning unit. In embodiments where an electrochemical cell produces both oxygen and hydrogen, hydrogen can be introduced into a pure or conditioned oxygen stream by mixing hydrogen into the oxygen stream between the electrochemical cell and the point of application to the tissue.

Mixing of gases can be achieved by a variety of techniques, including direct mixing by metering and passive mixing by diffusion. In the case of direct mixing to condition an oxygen stream, one stream (ambient air) may be injected into the oxygen stream by means of a controllable or tuned orifice (a metering valve, e.g.) and the combined streams may become mixed and, at equilibrium, achieve desired oxygen partial pressures (pO₂). In certain applications a proportional valve having an electronically controlled output flow may be desirable; in other applications, an on-off valve or pump, either having electrical actuation per a specified duty cycle, may be preferred.

Alternatively, direct mixing may involve injecting a stream of pure hydrogen into the oxygen stream by means of a controllable or tuned orifice (a metering valve, e.g.) and the combined streams may become mixed and, at equilibrium, may achieve the desired oxygen and/or hydrogen partial pressures (pO₂, pH₂). In a simpler embodiment, the conduit of each stream may join together or may join together in a mixing box, with the ratio of gases being set by the relevant electrochemical oxygen generator, or, in the case of oxygen, by an oxygen conditioning unit. In certain applications a proportional valve, having an electronically controlled output flow, may be desirable, and in others an on-off valve or pump, either having electrical actuation per a specified duty cycle, may be preferred. In another embodiment, a downstream H₂ sensor which gives feedback control may be applied to one or more of the following: the electrolyzer generating H₂, valves in the H₂ stream or any element in the oxygen stream. This allows the adjustment to the desired percentage of oxygen or hydrogen in the resulting outlet stream.

In passive mixing, the two streams being mixed may be separated by a permeable material which allows for mixing of the permeating gases in the oxygen stream. In this case, the permeable materials may be a plastic film, gas permeable tube or a porous phase separator, for instance. Any form factor which supports separation of the two mixing phases and embodies a permeable material interposed may suffice. Permeable materials may be selected for optimal characteristics, for instance, many rubbers have substantial differences in both solubility and diffusivity of various gases and so may be selected for preferred transport of a particular gas over another.

Optimal function of the mixing system may require the incorporation of a gas concentration sensor which can signal a digital or analog control system to alter an operating characteristic in the system and thereby attain and maintain the desired pO₂ and/or pH₂ levels. In this case, deviation from the desired pO2 or pH2 setpoint would cause a change to the mixing control setting, per standard proportional-integral-derivative or on-off process control loop implementation.

One embodiment which is disclosed herein involves the use of a permeation tube comprised of elastomeric material in conjunction with an electronically-actuated mechanical control which modifies the physical dimensions of the tube. For a tubular geometry, the rate of mixing per unit length is dictated by the partial pressure difference of each gas species across the tube wall, the thickness of the tube wall, and the permeability of the material, a bulk property which may or may not vary significantly with elastic deformation. For increased linear stretch of the elastomeric tube, the overall length increases and the wall thickness decreases, as the elastomer volume is ideally conserved, and these changes may result in generally better dilution of the interlumen gas, as permeation rates are higher when the permeation phase is thinner and when the surface area available for exchange is greater.

Another embodiment of the invention includes the use of multiple permeation tubes, with or without the actuated tube stretching, each individually addressable by electronically-controlled valves, so that a wide range of permeation rates may be more readily attained. The electronic feedback from the sensor tells a manifold box which outlet(s) to send the gas stream through to have the proper surface area for gas exchange.

Another embodiment of the invention includes the programmatic current control of the electrochemical cell current as an output from the digital or analog process control loop, thereby allowing for a simplified system in some cases where a fixed tube geometry or static valve orifice size is preferred.

Any of the foregoing systems could include flow sensors, pressure sensors or gas partial pressure sensors at a variety of points in the streams and incorporate those into feedback control steams to adjust the flow rate and gas composition as desired. Examples of placement of oxygen and hydrogen sensors may depicted in some embodiments, but other placements are possible and desirable.

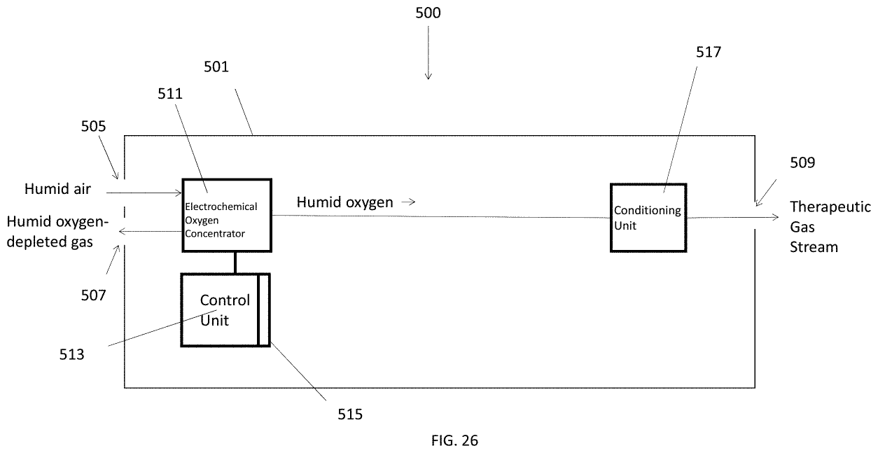

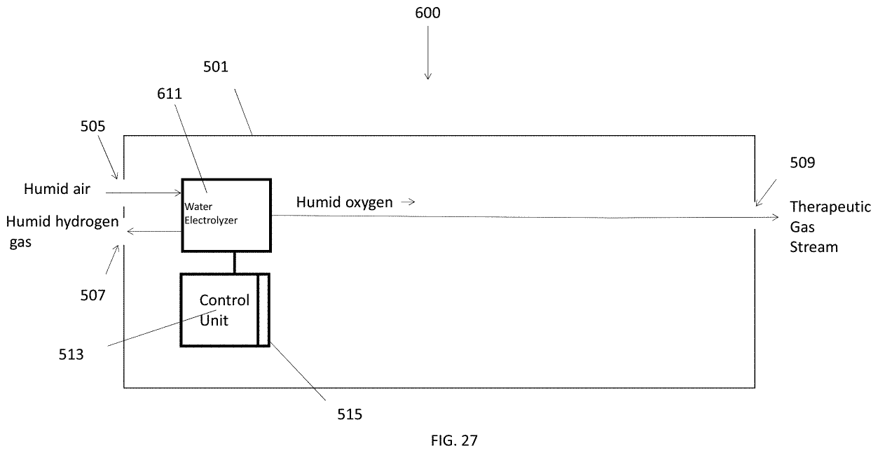

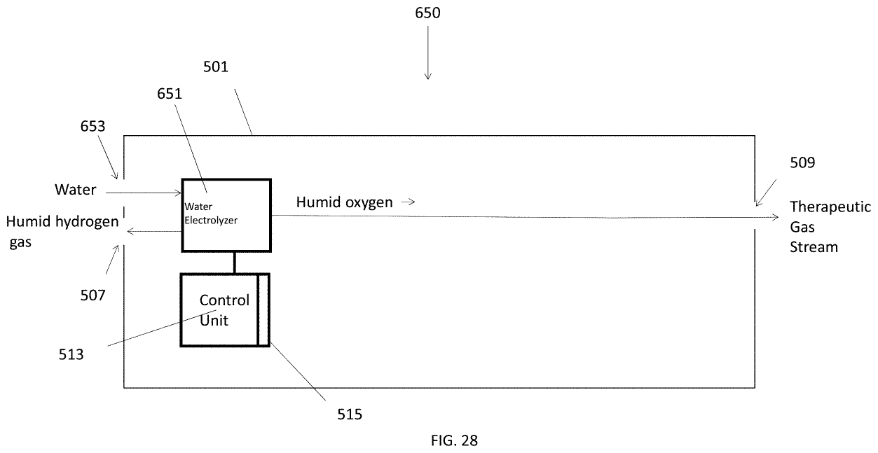

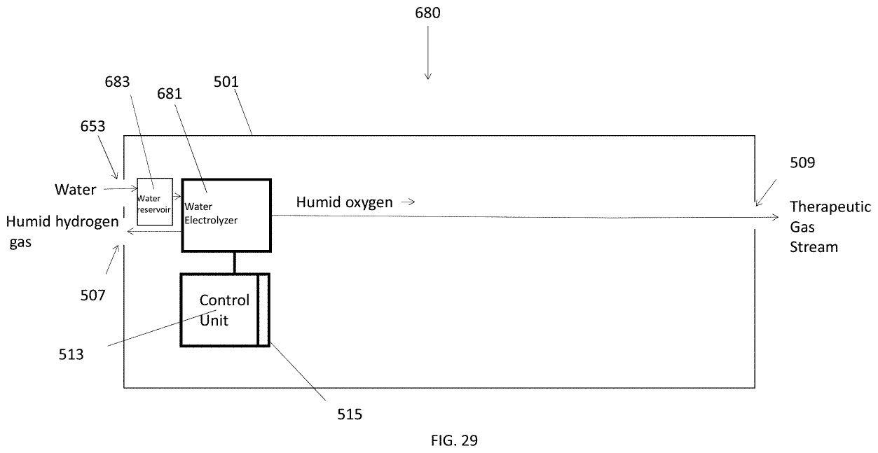

Referring now to FIG. 26, there is schematically shown an embodiment of a device for generating a therapeutic gas according to the present invention, the device being represented generally by reference number 500. (For simplicity and clarity, certain components of device 500 that are not critical to the understanding of the present invention are either not shown or described herein or are shown and/or described herein in a simplified manner.)

Device 500 may comprise a housing 501. Housing 501, in turn, may be shaped to include an inlet 505, which may be used to admit humid ambient air into housing, and may also be shaped to include an outlet 507, which may be used as an egress for humid oxygen-depleted gas, and an outlet 509, which may be used as an egress for a generated therapeutic gas.

Device 500 may further comprise an electrochemical oxygen concentrator (EOC) 511. EOC 511 may be disposed within housing 51 and may be used to release a stream of humid oxygen from humid ambient air.

Device 500 may further comprise a control unit 513 and a power supply 515, both of which may be disposed within housing 501. Control unit 513 may condition battery power and may send set current to EOC 511. In addition, control unit 513 may include data logging and could include a microcontroller and an electronic circuit element to achieve those functions. Power supply 515 may comprise a rechargeable or non-rechargeable battery of any chemistry or form factor.

Device 500 may further comprise a conditioning unit 517, which may be optional, disposed within housing 501. Conditioning unit 517 may be fluidly coupled to the output of EOC 511 and may be used to dilute the pure oxygen stream discharged from EOC 511, for example, by mixing the pure oxygen stream with ambient air that has entered housing 501 through inlet 505 to achieve a therapeutically desirable oxygen concentration.

An alternative to device 500 is schematically shown in FIG. 27 as device 600. (For simplicity and clarity, certain components of device 600 that are not critical to the understanding of the present invention are either not shown or described herein or are shown and/or described herein in a simplified manner.) Device 600 differs notably from device 500 in that device 600 may include a water electrolyzer 611, instead of an electrochemical oxygen concentrator 511, and also does not include structure corresponding to conditioning unit 517.

Another alternative to device 500, which alternative is similar to device 600, is schematically shown in FIG. 28 as device 650. (For simplicity and clarity, certain components of device 650 that are not critical to the understanding of the present invention are either not shown or described herein or are shown and/or described herein in a simplified manner.) Device 650 differs notably from device 600 in that device 650 may include a water electrolyzer 651 that has an internal refillable water reservoir that can be refilled through a port 653.

Still another alternative to device 500, which alternative is similar to device 650, is schematically shown in FIG. 29 as device 680. (For simplicity and clarity, certain components of device 680 that are not critical to the understanding of the present invention are either not shown or described herein or are shown and/or described herein in a simplified manner.) Device 680 differs notably from device 650 in that device 680 may include a water electrolyzer 681 that has a separate internal refillable water reservoir 683 that can be refilled through a port 653.

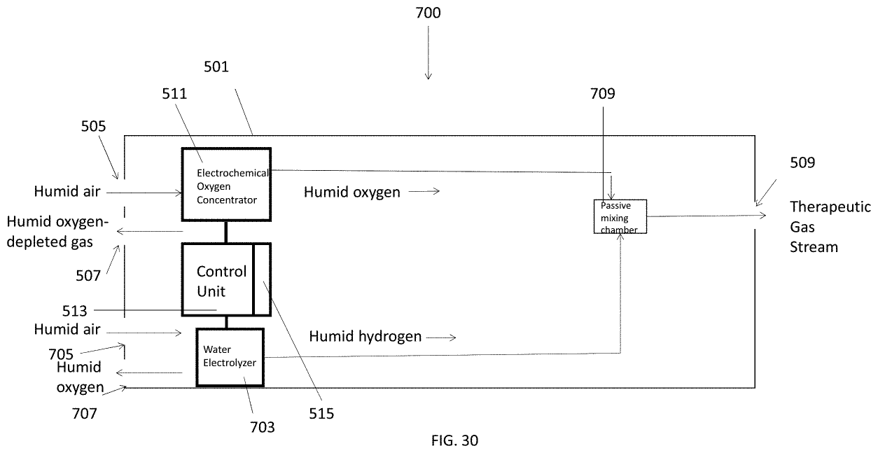

Yet another alternative to device 500 is schematically shown in FIG. 30 as device 700. (For simplicity and clarity, certain components of device 700 that are not critical to the understanding of the present invention are either not shown or described herein or are shown and/or described herein in a simplified manner.) Device 700 differs notably from device 500 in that device 700 additionally includes a water electrolyzer 703, which may receive humid air through an inlet 705 and release generated oxygen through an outlet 707. The oxygen outputted from EOC 511 and the hydrogen outputted from water electrolyzer 703 may be mixed in an optional passive mixing chamber 709.

It may be noted that the combination of an electrochemical oxygen concentrator generating oxygen with an electrolyzer for generating hydrogen could enable the electrolyzer to be very small comparatively in size and H₂ generating capability. This may be quite advantageous.

It may also be noted that an electrochemical oxygen concentrator and an electrolyzer may be controlled by either one or more control units.

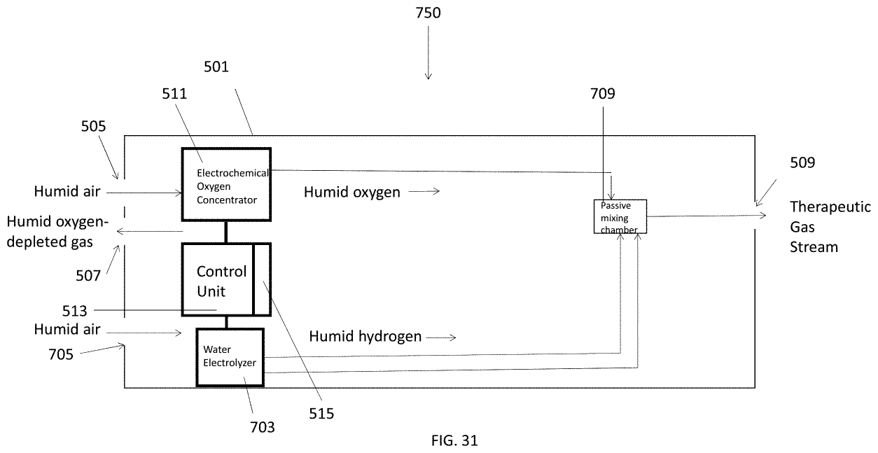

Still yet another alternative to device 500 is schematically shown in FIG. 31 as device 750. (For simplicity and clarity, certain components of device 750 that are not critical to the understanding of the present invention are either not shown or described herein or are shown and/or described herein in a simplified manner.) Device 750 is similar to device 700, except that, in device 750, both the hydrogen and the oxygen outputted from electrolyzer 703 may be included in the generated treatment gas.

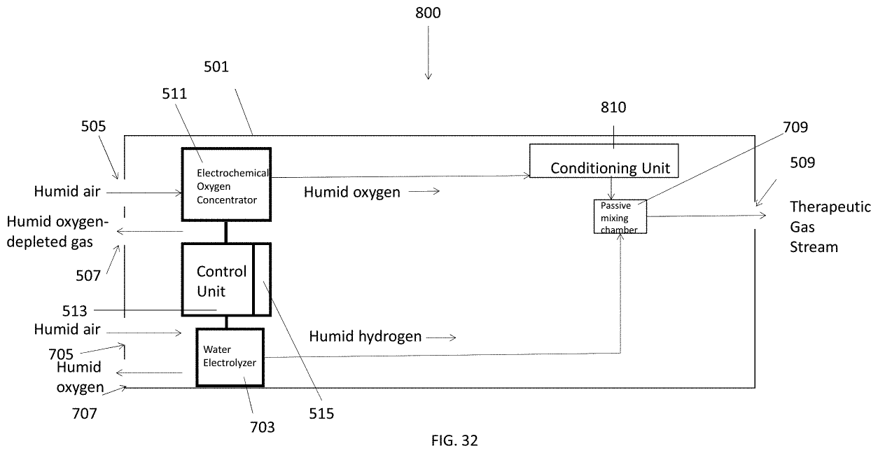

Still a further alternative to device 500 is schematically shown in FIG. 32 as device 800. (For simplicity and clarity, certain components of device 800 that are not critical to the understanding of the present invention are either not shown or described herein or are shown and/or described herein in a simplified manner.) Device 800 is similar to device 750, except that device 800 may further include a conditioning unit 810 for the oxygen gas generated by EOC 511.

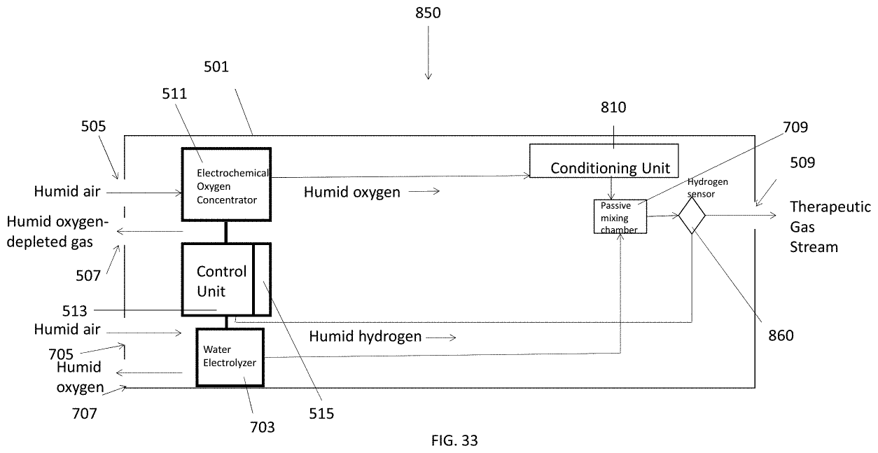

Still a further alternative to device 500 is schematically shown in FIG. 33 as device 850. (For simplicity and clarity, certain components of device 850 that are not critical to the understanding of the present invention are either not shown or described herein or are shown and/or described herein in a simplified manner.) Device 850 is similar to device 800, except that device 850 may further include a hydrogen sensor 860 that it coupled to control unit 513 to regulate the operation of water electrolyzer 703.

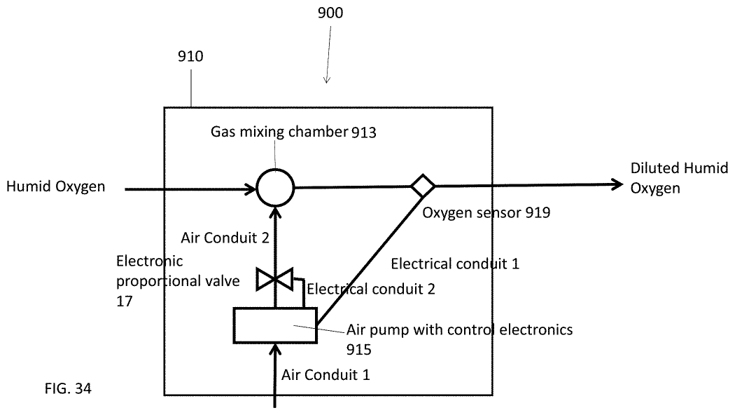

Referring now to FIG. 34, there is schematically shown an embodiment of a conditioning unit that may be suitable for use in a device for generating a therapeutic gas, the conditioning unit being represented generally by reference numeral 900. (For simplicity and clarity, certain components of conditioning unit 900 that are not critical to the understanding of the present invention are either not shown or described herein or are shown and/or described herein in a simplified manner.)

Conditioning unit 900, which may be suitable for use as conditioning unit 517 in device 500 or as conditioning unit 810 in device 800, may comprise a housing 910. A gas mixing chamber 913 may be disposed within housing 910 and may be used to receive through an inlet (not shown) humid oxygen, for example, from an electrochemical oxygen concentrator or water electrolyzer. Gas mixing chamber 913 may also receive ambient air, which may enter housing 910 through an inlet (not shown) and thereafter be pumped using a pump 915 to gas mixing chamber 913. An electronic proportional valve 917 may optionally be fluidly interposed between pump 915 and gas mixing chamber 913. The gas leaving gas mixing chamber 913 may be conducted to an oxygen sensor 919. Oxygen sensor 919 is electrically coupled to pump 915 and may regulate the operation of pump 915 based on the oxygen concentration sensed by oxygen sensor 919. The diluted humid oxygen may then leave housing 910 through an outlet (not shown).

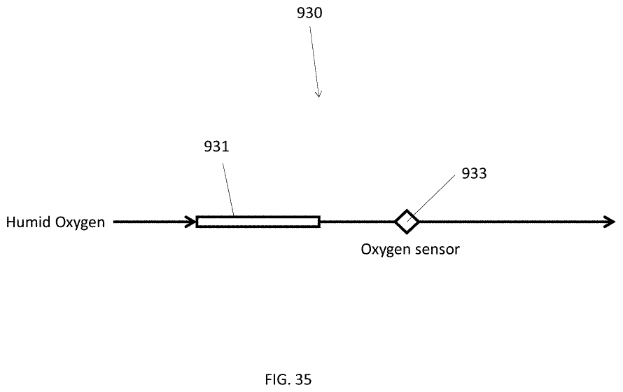

An alternative embodiment of a conditioning unit is shown in FIG. 35 and is represented generally as conditioning unit 930. (For simplicity and clarity, certain components of conditioning unit 930 that are not critical to the understanding of the present invention are either not shown or described herein or are shown and/or described herein in a simplified manner.)

Conditioning unit 930 may comprise a length of gas permeable tubing 931 whose inlet is fluidly coupled to the output of the oxygen generating device (e.g., EOC, water electrolyzer). As humid oxygen passes through tubing 931, it mixes with ambient air that permeates through the wall of tubing 931. Conditioning unit 930 may also include an oxygen sensor 933.

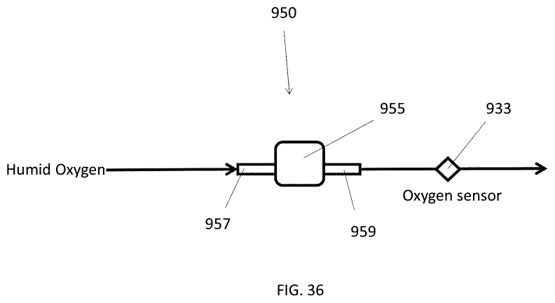

Yet another alternative embodiment of a conditioning unit is shown in FIG. 36 and is represented generally as conditioning unit 950. (For simplicity and clarity, certain components of conditioning unit 950 that are not critical to the understanding of the present invention are either not shown or described herein or are shown and/or described herein in a simplified manner.)

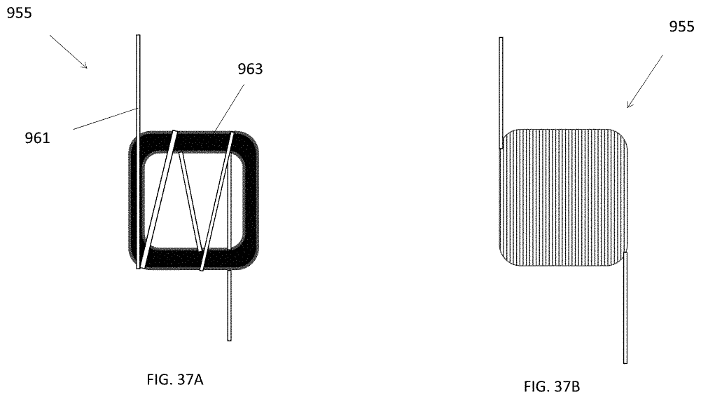

Conditioning unit 950 may comprise the combination of a high gas permeable structure 955 interposed between and fluidly connected to two low gas permeable structures 957 and 959. High gas permeable structure 955 may comprise a long length of gas permeable tubing coiled around a frame (see examples in FIGS. 37A and 37B in which tubing 961 is wrapped around frame 963 at a low pitch and at a high pitch, respectively). Conditioning unit 930 may also include an oxygen sensor 933.

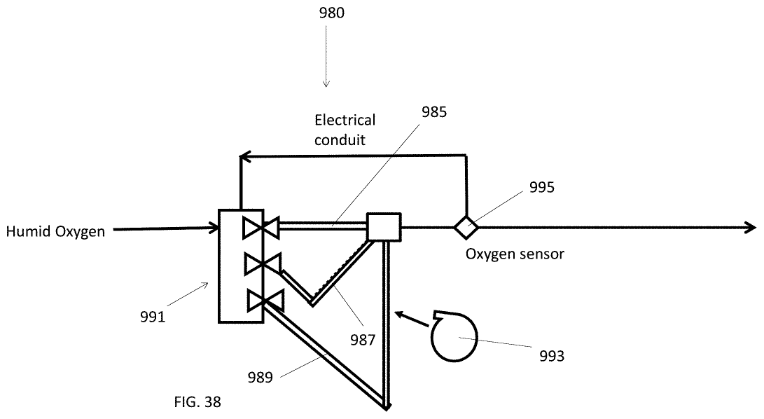

Still yet another alternative embodiment of a conditioning unit is shown in FIG. 38 and is represented generally as conditioning unit 980. (For simplicity and clarity, certain components of conditioning unit 980 that are not critical to the understanding of the present invention are either not shown or described herein or are shown and/or described herein in a simplified manner.)

Conditioning unit 980 may comprise three lengths of gas permeable tubing 985, 987 and 989, with tubing 985 being the shortest of the three and with tubing 989 being the longest of the three. Conditioning unit 980 may further comprise a manifold 991 with electronic valving and control electronics to selectively direct humid oxygen arriving from an oxygen generating device to one or more of tubings 985, 987, and 989 depending on how much dilution of the oxygen is desired. The longer the tubing, the greater the dilution. Conditioning unit 980 may further comprise an oxygen sensor 995 locating downstream, which may be connected to manifold 991 to provide feedback control. An air blower 993 may also be optionally included to increase the flow of air across tubings 985, 987, and 989.

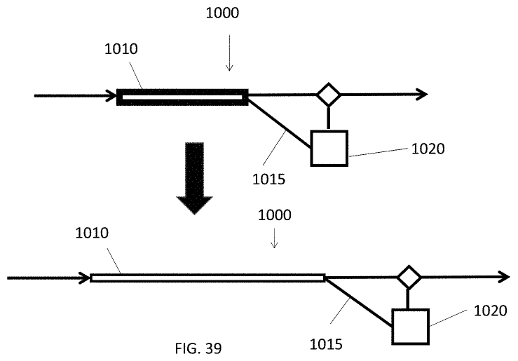

A further alternative embodiment of a conditioning unit is shown in FIG. 39 and is represented generally as conditioning unit 1000. (For simplicity and clarity, certain components of conditioning unit 1000 that are not critical to the understanding of the present invention are either not shown or described herein or are shown and/or described herein in a simplified manner.)

Conditioning unit 1000 may comprise a length of gas permeable, reversibly extensible tubing 1010. Tubing 1010 may be porous but need not be. A suitable material for tubing 1010 may be silicone. Conditioning unit 1000 may additionally comprise a stretching unit for selectively stretching tubing 1010. The stretching unit may be, for example, the combination of a belt/cord 1015 and a winder 1020, which may be controlled electronically. As can be seen in FIG. 39, as tubing 1010 is stretched, its wall thickness decreases, thereby increasing its gas permeability.

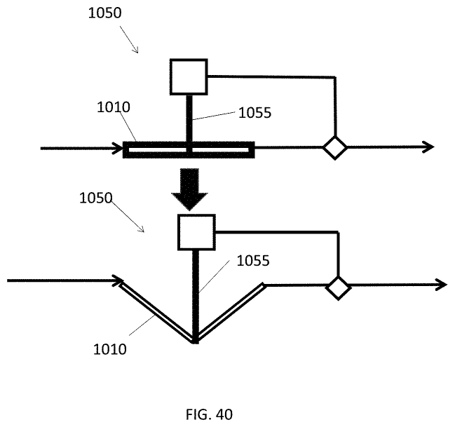

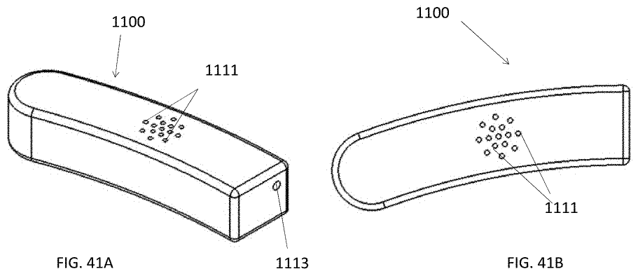

Still a further alternative embodiment of a conditioning unit is shown in FIG. 40 and is represented generally as conditioning unit 1050. (For simplicity and clarity, certain components of conditioning unit 1050 that are not critical to the understanding of the present invention are either not shown or described herein or are shown and/or described herein in a simplified manner.)

Conditioning unit 1050 differs from conditioning unit 1000 in that a rigid plunger 1055 with a collar around tubing 1010 is used to stretch tubing 1010, thereby causing tubing 1010 to lengthen and its walls to become more thin.

Referring now to FIGS. 41A and 41B, there are shown various views of an alternative embodiment of a housing designed for use in a device for electrochemically generating a gas, the housing being represented generally as housing 1100. (For simplicity and clarity, certain components of housing 1100 that are not critical to the understanding of the present invention are either not shown or described herein or are shown and/or described herein in a simplified manner.)

Housing 1100, which may be designed to be worn over a user's ear, may include a plurality of inlets 1111 and a single outlet 1113.

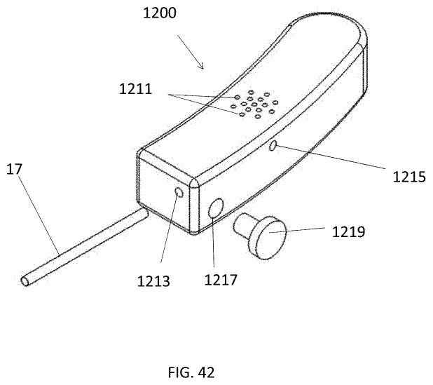

An alternative housing is shown in FIG. 42 and is represented generally as housing 1200. (For simplicity and clarity, certain components of housing 1200 that are not critical to the understanding of the present invention are either not shown or described herein or are shown and/or described herein in a simplified manner.) Housing 1200, which may also be worn over a user's ear, may include a plurality of inlets 1211 and a first outlet 1213, which may be used to receive tubing 17. Housing 1200 may further comprise a second outlet 1215, which may be used to vent a secondary gas, such as hydrogen, and a third outlet 1217, which may be used to receive water. A plug 1219 may be used to close third outlet 1217.

It is to be understood that, although the various different types of gas supply devices disclosed herein have been discussed in the context of providing a therapeutic gas to an eye, the present invention is not limited to such an application. Accordingly, for example, such gas supply devices may be used to provide a therapeutic gas for administration to an ear, as in U.S. patent application Ser. No. 17/408,396, or may be used to provide one or more therapeutic gases to a biological sample or cell implant, or may be used for entirely non-therapeutic purposes.

The following example is given for illustrative purposes only and is not meant to be a limitation on the invention described herein or on the claims appended hereto.

### Example

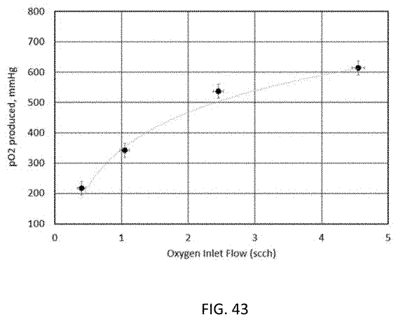

An example of passive oxygen conditioning to lower partial pressure (concentration) is provided herein. An electrochemical oxygen generator (Giner Life Sciences, Inc., Newton, Mass., 0.3-cm² cathode water vapor feed electrolyzer) was operated at various currents (19.9, 11.1, 4.82 and 1.92 mA) to achieve a range of output oxygen flows. The oxygen outlet was connected by low permeability tubing ( 1/16″ OD, 0.028″ wall polyetheretherketone) to a mass flow meter (Alicat MW-0.5SCCM-D) which measured oxygen mass flow and pressure. The outlet of the mass flow meter was connected to a polydimethylsiloxane (silicone) rubber tube of 11 cm exposed length, 0.064 cm inner diameter and 0.028 cm wall thickness (McMaster-Carr part 51845K67). The outlet of the silicone tube was connected to a flow-through oxygen sensor (PreSens Precision Sensing Model FTC-PSt3 with EOM-O2-mini electro-optical transducer). The flow and oxygen partial pressure values were measured and recorded by serial connection to a computer after sufficient duration to achieve steady-state. The graph of FIG. 43 indicates the regular trend in outlet oxygen partial pressure and that it compares well to what is predicted by a well-mixed permeation model.

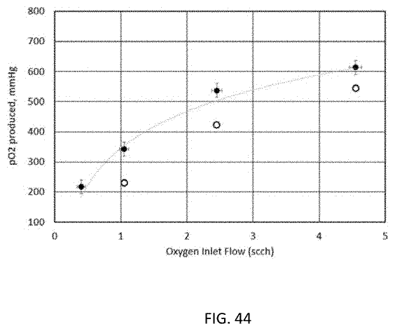

The experiment was repeated when the silicone tube was stretched by 150% in length. Stretching causes thinning of the walls and narrowing of the internal diameter, in addition to an increase in the length of the tubing available for permeation exchange with ambient air; the wall thickness and diameter during stretching were not measured. The open circles shown in FIG. 44 demonstrate the increased dilution achieved across a range of flows when the tube was stretched (150%) versus control (100%) in the closed circles. The effect is completely reversible when allowing the tube to relax to its original state.

Additional objects, features, and advantages of the invention are set forth below.

It is an object of the present invention to provide a novel method and system for modifying the fluid environment of an eye, such as by providing oxygen and/or hydrogen gas to the eye, and protecting the eye from the ambient environment.

Where hydrogen is delivered to tissue such as the eye, such hydrogen may have anti-inflammatory, antioxidant, antiapoptotic properties and may speed and improve the healing of the eye by itself or synergistically with oxygen. The hydrogen may have a protective effect against the negative consequences of elevated (above ambient) oxygen. Oxygen, by providing a reactant for the creation of cellular energy, can improve cell healing, cell health and the aspects of cellular metabolism and repair.

It is another object of the present invention to provide a method and system as described above that addresses the shortcoming associated with existing post-surgical eye shields for protecting the eye from particulates and irritants in the ambient environment and inadvertent rubbing, etc.

The above-described system may sometimes be referred to herein as an Eye Oxygenation Device with hydrogen (EOD-H).

It is still another object of the present invention to provide an EOD-H as described above that is compact, that has a minimal number of parts, that is inexpensive to manufacture, that is electrically efficient, that is reliable, and that is easy to operate. Preferably, the device is simple and low cost, is quiet, is hands-free, is portable and location independent, is comfortable, and is discreet.

Therefore, according to one aspect of the invention, there is provided an EOD-H, the device comprising (a) an electrochemical oxygen generator (EOG); (b) control electronics for controlling the EOG's operation; (c) a power source coupled to the EOG and the control electronics for controlling the EOG's operation; (d) means for delivering a pure, controlled humidity oxygen stream to the eye; (e) means for directing humid gas out of the area above the eye; (f) an eye cover; and (g) one or more housing components comprising some or all of the aforementioned components.

In another aspect of the invention the EOD-H may comprise two electrochemical oxygen generators with one configured to provide ambient or enriched oxygen concentration to the eye and the second an electrochemical oxygen generator configured to produce a lower volumetric flow rate than the first. In a preferred embodiment the first electrochemical device would be an oxygen concentrator producing oxygen and the second electrochemical device would be an electrolyzer producing lower volumetric flow rate of oxygen than the first device and also producing the stoichiometric volume of hydrogen as dictated by the electrolysis electrochemical reaction and delivering this mixture to the area above the eye. In this embodiment the oxygen from the second device may be included in the overall gas stream or discarded to simplify gas plumbing. A further preferred embodiment is one in which the two electrochemical devices are operated in such a mode that the combination of the outlet streams leads to a combined gas mixture which is 0.1-3.9% hydrogen by volume with the balance oxygen with water vapor. This latter preferred embodiment where hydrogen generation in the mixture is below the hydrogen flammability limit of 4% in oxygen. Combining the gas streams from two electrochemical devices in this manner allows only a safe amount of hydrogen in oxygen to be made. In electrolysis, as is known in the art, hydrogen is produced at twice the molar volume of oxygen. Notwithstanding the above, the present invention could be used to generate any ratio of O₂/N₂/H₂ if proper safety considerations known in the field are applied.

In a more detailed feature of the invention, the device may be designed for providing oxygen, hydrogen or mixture gas to the right eye. Oxygen, hydrogen or mixture gas is sometimes referred to as a therapeutic gas.

In a more detailed feature of the invention, the device may be designed for providing a therapeutic gas to the left eye.

In a more detailed feature of the invention, the device may be designed for providing a therapeutic gas to both eyes.

In a more detailed feature of the invention, the eye cover may be a shield that fits over one eye.

In a more detailed feature of the invention, the shield is held in place via medical tape or other suitable means.

In a more detailed feature of the invention, the eye cover may be a pair of goggles that fits over both eyes.

In a more detailed feature of the invention, the goggles are held in place via an elastic band; an adjustable inelastic band; or other suitable means.

In a more detailed feature of the invention, the eye cover may be designed to fit between the eye and one or both eyelids, such as a contact lens bandage that fits directly onto the eye.

In a more detailed feature of the invention, the contact lens bandage is held in place by sticking to the tear fluid that coats the surface of the eye and having a curvature similar to the curvature of the cornea.

In a more detailed feature of the invention, the eye cover may be optically clear, transparent, and translucent.

In a more detailed feature of the invention, the eye cover may be tinted to varying degrees of transparency and translucence.

In a more detailed feature of the invention, the eye cover may have a gas impermeable foam around the perimeter where the eye cover contacts the orbital bones surrounding the eye. This impermeable foam provides a comfortable, gas tight fit to allow the EOD-H to raise the partial pressure of oxygen in the area between the eye cover and the eye.

In a more detailed feature of the invention, the eye cover may have a gas permeable filter to allow excess gas to leave the area between the eye cover and the eye, preventing humidity and pressure build-up.

In a more detailed feature of the invention, the device may comprise an electronics housing that is discrete from the eye cover, and the electronics housing may be designed to fit behind the ear, similar to a behind-the-ear hearing aid.

In a more detailed feature of the invention, the device may comprise an electronics housing that is discrete from the eye cover, and the electronics housing may be designed to mount onto a pair of glasses.

In a more detailed feature of the invention, the device may comprise an electronics housing that is discrete from the eye cover, and the electronics housing may be designed to rest against the scalp, being held in place by a headband, hat, or similar.

In a more detailed feature of the invention, the EOG may be incorporated into the eye cover for direct delivery of gas to the area above the eye.

In a more detailed feature of the invention, the EOG may be annular in shape when incorporated into the contact lens bandage eye cover to allow for minimal impact to the field of vision.

In a more detailed feature of the invention, the EOG may be incorporated into the electronics housing.

In a more detailed feature of the invention, the electronics housing may be connected to an oxygen delivery port integrated into the eye cover.

In a more detailed feature of the invention, the device may comprise an electronics housing, and the electronics housing may be detachably coupled to a sterilized, disposable eye cover delivery port via a disposable tube set.

In a more detailed feature of the invention, the device may comprise an electronics housing, and the electronics housing may be permanently coupled to the eye cover delivery port via a permanent tube set.

In a more detailed feature of the invention, the tube set may be made of rigid tubing, similar to that used in Behind-The-Ear Hearing aids, to hold the electronics housing in place behind the ear.

In a more detailed feature of the invention, the tube set may be made of soft tubing to allow the electronics housing to be positioned on the scalp where comfortable.

In a more detailed feature of the invention, the device may comprise an electronics housing, and the electronics housing may be designed to be integrated into the frame of the eye cover goggles.

In a more detailed feature of the invention, the device may comprise an electronics housing, and the electronics housing may be designed to be integrated into the frame of a pair of glasses.

In a more detailed feature of the invention, the control electronics may comprise an on/off switch that may be used to control when the device operates.

In a more detailed feature of the invention, the control electronics may include a simple circuit that begins operation when the power source is installed and ends operation when the power source runs out of power and/or is removed from the device.

In a more detailed feature of the invention, the control electronics may include a circuit that provides a constant current to the EOG.

In a more detailed feature of the invention, the control electronics may include a circuit that provides a constant voltage that is converted to a current and provided to the EOG.

In a more detailed feature of the invention, the control electronics may include circuitry that decreases applied current to the EOG and, hence, oxygen production when the power source reaches a low level in order to extend oxygen production life.

In a more detailed feature of the invention, the control electronics may incorporate power monitoring circuitry and a low battery alarm that may provide an audible, visual or motion signal to the user, caregiver or physician.

In a more detailed feature of the invention, the control electronics may incorporate an induction circuit to transmit power from the electronics housing to the EOG embedded in a contact lens eye cover.

In a more detailed feature of the invention, the control electronics may interface with one or more sensors including, but not limited to, pressure sensors, humidity sensors, voltage sensors, gas sensors, flow sensors, and accelerometers. The control electronics may use sensors to provide feedback control to control some aspect of the operation of the EOD-H. These aspects may include on/off or current level.

In a more detailed feature of the invention, the EOD-H may have a switch that allows a physician to set one of several preprogrammed flow rates to adjust the device based on a desired flow rate dependent upon a patient's eye condition or body size. The control electronics may include an electronic mechanism to provide the current set points for such flow rates.

In a more detailed feature of the invention, the control electronics may include a microprocessor.

In a more detailed feature of the invention, the control electronics may include analog electronics without the use of a microprocessor.

In a more detailed feature of the invention, the control electronics may provide a higher start-up current for a period of time to flush a tubing system and/or the area between the eye cover and the eye.

In a more detailed feature of the invention, the control electronics may provide for intermittent provision of oxygen to meet a therapeutic need or to conserve energy.

In a more detailed feature of the invention, the device may be powered by a disposable battery.

In a more detailed feature of the invention, the electronics housing may have a mechanism for accessing the battery for replacement.

In a more detailed feature of the invention, the device may be powered by a rechargeable battery.

In a more detailed feature of the invention, the electronics housing may include a mechanism for recharging the battery.

In a more detailed feature of the invention, the control electronics may incorporate capacitors to accumulate charge as the power source.

In a more detailed feature of the invention, the EOG or EOGs may produce oxygen and/or hydrogen continuously.

In a more detailed feature of the invention, the EOG(s) may produce oxygen and/or hydrogen intermittently, cycling on and off.

In a more detailed feature of the invention, the EOG(s) may cycle on and off at different frequencies to produce gas(es) at a desired flow rate.

In a more detailed feature of the invention, the gas delivery port path may be designed for direct oxygen delivery to the eye.

In a more detailed feature of the invention, the gas delivery path may follow the periphery of the eye cover, with multiple ports around the periphery.

In a more detailed feature of the invention, the gas delivery path may follow the periphery of the eye cover, with a single port around the periphery.

In a more detailed feature of the invention, the gas delivery port path may be designed to interact with an air ingress port from outside the eye cover to provide an oxygen-enriched gas stream that is not pure oxygen. The interaction may include drawing air in via a venturi effect or other convective or diffusive means.

In a more detailed feature of the invention, the gas ingress port path may be designed for oxygen to be delivered to the eye in a vortex.

In a more detailed feature of the invention, the gas ingress port path may be designed for laminar flow oxygen delivery to the eye.

In a more detailed feature of the invention, the oxygen ingress port path is designed for turbulent flow oxygen delivery to the eye.

In a more detailed feature of the invention, the eye cover may include a gas egress port for gas release from the area over the eye. The gas egress port path may connect the gas delivery location to the ambient environment.

In a more detailed feature of the invention, the gas delivery path and the gas egress path may be the same length.

In a more detailed feature of the invention, the gas delivery path and the gas egress path may be different lengths.

In a more detailed feature of the invention, the flow path of the gas egress port may pass through a cathode support as a means of reactant delivery to the electrochemically-active components.

In a more detailed feature of the invention, the flow path of the gas egress port through the device earpiece may pass adjacent to a vapor transport membrane (VTM). The VTM may separate the hydrogen produced by electrolysis from the oxygen in the gas egress port, allowing the carried vapor to migrate across the membrane to be used as a reactant at the cathode.

In a more detailed feature of the invention, the flow path of the gas egress port may comprise a blower to assist in removal of gas from the area over the eye.

In a more detailed feature of the invention, the flow path of the gas egress port may comprise a flap to create convective flow. The flap may be activated by normal head and jaw movement.

In a more detailed feature of the invention, the flow path of the gas egress port may comprise a desiccant to prevent condensate build-up.

In a more detailed feature of the invention, the desiccant may be adjacent to the gas egress port path.

In a more detailed feature of the invention, the desiccant may be replaceable by the user. In a more detailed feature of the invention, the eye cover may have a pressure relief valve on the gas egress path for emergency pressure release from the area over the eye.

In a more detailed feature of the invention, the electrochemical oxygen generator(s) may be a self-regulating electrochemical gas generator with intrinsic pressure relief as in U.S. Pat. No. 10,557,691.

In a more detailed feature of the invention, the eye cover may have a medication delivery port allowing medicine to be delivered to the eye without removing the eye cover.

In a more detailed feature of the invention, the eye cover may have an instrument port allowing medical professionals to perform surgical revisions in the eye without removing the eye cover.

In a more detailed feature of the invention, the device earpiece may have a condensate drop out port for removing built-up liquid from the area over the eye.

In a more detailed feature of the invention, the various port paths may be a void in the eye cover.

In a more detailed feature of the invention, the various port paths may comprise a tube integrated in the eye cover.

In a more detailed feature of the invention, the quantity of oxygen and its flow rate may be defined by the current set point of the electrochemical oxygen generator and can be varied depending on the application.

In a more detailed feature of the invention, the quantity of oxygen and its flow rate may be defined by the current set point of the electrochemical oxygen generator and can be varied depending on the application.

In a more detailed feature of the invention, the quantity of oxygen and hydrogen and their flow rates may be defined by the current set point of the electrochemical oxygen generator and can be varied independently depending on the application. These settings will define a gas ratio or composition.

In a more detailed feature of the invention, the first electrochemical oxygen generator may be a water electrolyzer.

In a more detailed feature of the invention, the first electrochemical oxygen generator may be an electrochemical oxygen concentrator.

In a more detailed feature of the invention, the second electrochemical oxygen generator may be a water electrolyzer.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. A system for delivering a therapeutic gas to the exterior of an eye, the system comprising:
(a) a gas source for supplying a treatment gas; and
(b) a gas delivery device, the gas delivery device being wearable over and substantially covering an exterior of an eye, the gas delivery device being fluidly coupled to the gas source to receive the treatment gas from the gas source, the gas delivery device comprising at least one gas outlet for use in delivering the treatment gas to the exterior of the eye.

2. The system as claimed in claim 1 wherein the gas source comprises an electrochemical gas generating device.

3. The system as claimed in claim 2 wherein the electrochemical gas generating device comprises at least one of a water electrolyzer and an electrochemical oxygen concentrator.

4. The system as claimed in claim 3 wherein the electrochemical gas generating device comprises both a water electrolyzer and an electrochemical oxygen concentrator.

5. The system as claimed in claim 2 wherein the treatment gas comprises at least one of oxygen and hydrogen.

6. The system as claimed in claim 5 wherein the treatment gas comprises oxygen at a concentration above that in ambient air.

7. The system as claimed in claim 6 wherein the treatment gas comprises oxygen at a concentration below pure oxygen.

8. The system as claimed in claim 5 wherein the treatment gas comprises both oxygen and hydrogen.

9. The system as claimed in claim 8 wherein the treatment gas comprises hydrogen at a concentration below 4% by volume.

10. The system as claimed in claim 5 wherein the treatment gas comprises oxygen but not hydrogen.

11. The system as claimed in claim 5 wherein the treatment gas comprises hydrogen but not oxygen.

12. The system as claimed in claim 1 wherein the gas delivery device comprises an eye shield.

13. The system as claimed in claim 12 wherein the gas source comprises a housing wearable over an ear.

14. The system as claimed in claim 12 wherein the gas source comprises a housing adapted to be clipped onto goggles.

15. The system as claimed in claim 1 wherein the gas delivery device comprises goggles.

16. The system as claimed in claim 15 wherein the goggles comprise fluid conduits and wherein the gas source is mountable on the goggles in fluid communication with the fluid conduits.

17. The system as claimed in claim 1 wherein the gas delivery device comprises a contact lens bandage.

18. The system as claimed in claim 1 wherein the gas delivery device comprises an eye shield and a foam gasket and wherein the eye shield has an inlet for receiving the therapeutic gas from the gas source.

19. The system as claimed in claim 1 wherein the gas delivery device comprises an eye shield and a foam gasket and wherein the foam gasket has an inlet for receiving the therapeutic gas from the gas source.

20. A device for generating a therapeutic gas, the device comprising:
(a) a housing;
(b) an electrochemical oxygen generating device disposed within the housing; and
(c) a conditioning unit disposed within the housing for diluting oxygen generated by the electrochemical oxygen generating device.

21. The device as claimed in claim 20 wherein the electrochemical oxygen generating device comprises a water electrolyzer.

22. The device as claimed in claim 20 wherein the electrochemical oxygen generating device comprises an electrochemical oxygen concentrator.

23. The device as claimed in claim 20 wherein the conditioning unit comprises a length of gas permeable tubing.

24. The device as claimed in claim 23 wherein the gas permeable tubing increases in gas permeability when stretched and wherein said conditioning unit further comprises a stretcher for the gas permeable tubing.

25. A device for generating a therapeutic gas, the device comprising:
(a) a housing;
(b) an electrochemical oxygen concentrator disposed within the housing;
(c) an electrolyzer disposed within the housing; and
(d) a mixing chamber for mixing outputs of the electrochemical oxygen concentrator and the electrolyzer.
